(19) United States
(12) Patent Application Publication
(10) Pub. No.: US 2020/0030716 A1
(43) Pub. Date: Jan. 30, 2020

(54) TASK SPECIFIC CHELATING IONIC LIQUIDS FOR REMOVAL OF METAL IONS FROM AQUEOUS SOLUTION VIA LIQUID/LIQUID EXTRACTION AND ELECTROCHEMISTRY

(71) Applicant: Massachusetts Institute of Technology, Cambridge, MA (US)

(72) Inventors: T. Alan Hatton, Sudbury, MA (US); Paul Brown, Boston, MA (US); Sahag Voskian, Cambridge, MA (US)

(21) Appl. No.: 16/522,224

(22) Filed: Jul. 25, 2019

Related U.S. Application Data
(60) Provisional application No. 62/703,166, filed on Jul. 25, 2018.

(51) Int. Cl. B01D 11/04; B01D 57/00; C07D 267/00

(52) U.S. Cl. CPC B01D 11/0492; C02F 1/26; C07D 267/00; B01D 57/00

(57) ABSTRACT

Disclosed are methods of extracting metal ions using ionic liquids (ILs), IL complexes, and mixtures comprising an IL and a metal-chelating group. Also disclosed are IL complexes, and mixtures comprising an IL and a metal-chelating group.

### RELATED APPLICATION

This application claims the benefit of priority to U.S. Provisional Patent Application Ser. No. 62/703,166, filed Jul. 25, 2018.

### BACKGROUND

The increased use of heavy metals and metalloids in industrial, agricultural and technological applications has led to their wide distribution and persistence in natural water bodies and soil [1, 2]. Elements such as lead, cadmium, nickel, mercury, zinc, arsenic and copper may cause multiple organ damage even at low exposure (maximum contaminant level, MCL, of lead is 0.006 mg/L [3]) and are therefore of public health significance [4]. Established technologies to remove metal ions from waste water are varied and include i) ion exchange resins [5, 6], which have high capacities and removal efficiencies, but often prove problematic to regenerate; ii) membrane filtration [7], which is low energy and high efficiency but has problems of fouling and sometimes requires the addition of a surfactant; iii) coagulation and flocculation [8, 9], which is not efficient and requires the use of polymers and/or further treatment; iv) flotation [10], which requires the use of surfactants; v) adsorption [11], where adsorbents are not always regenerable or are expensive (e.g., activated charcoal); vi) chemical precipitation [12-14], which is low cost but requires the use of a large amount of chemicals and can form sludges; vii) electrochemical treatment [15], which requires large capital investment; viii) solvent (liquid/liquid) extraction, which conventionally requires the use of volatile organic compounds (VOCs).

More recently, liquid/liquid extractions have been made possible by the development of ionic liquids. Ionic liquids (ILs) are simply salts that are liquid at room temperature. They typically consist of a bulky cation and a small halogenated anion. These salts provide a non-aqueous yet polar medium and therefore have unusual solvent properties. The first ILs designed for heavy metal extraction favorably partitioned metals bound to complexing agents [16], but by appending the cation with metal-ion ligating functional groups, selective extraction of solute metals was achieved directly [17-20]. These new functionalized ILs were named “task specific ILs”. However, removal of the metal ions from the IL has hardly been demonstrated and recyclability is therefore limited. To date the only possible process reported has been further washing of the IL with organic solvent [21]; a rather expensive and environmentally unfriendly approach. Accordingly, additional methods for extracting metal ions from aqueous solutions are needed.

### SUMMARY

Disclosed herein are methods of extracting metal ions using ionic liquids (ILs), IL complexes, and mixtures comprising an IL and a metal-chelating group.

In one aspect, the disclosure provides a method of removing metal cations from an ionic liquid mixture, comprising:

providing an ionic liquid mixture comprising an ionic liquid (IL), wherein the IL comprises a metal-chelating group, and a plurality of metal cations; and

applying an electrical potential to the ionic liquid mixture, thereby removing from the ionic liquid mixture the plurality of metal cations.

In another aspect, the disclosure provides a method of removing metal cations from an aqueous mixture, comprising:

providing an aqueous mixture comprising water and a plurality of metal cations;

contacting the aqueous mixture with an ionic liquid, wherein the IL comprises a metal-chelating group, thereby forming an ionic liquid mixture comprising the ionic liquid and the plurality of metal cations; and

applying an electrical potential to the ionic liquid mixture, thereby removing from the ionic liquid mixture the plurality of metal cations.

In another aspect, the disclosure provides a method of removing metal cations from an ionic liquid mixture, comprising:

providing an ionic liquid mixture comprising an ionic liquid, a metal-chelating group, and a plurality of metal cations; and

applying an electrical potential to the ionic liquid mixture, thereby removing from the ionic liquid mixture the plurality of metal cations.

In another aspect, the disclosure provides a method of removing metal cations from an aqueous mixture, comprising:

providing an aqueous mixture comprising water and a plurality of metal cations;

contacting the aqueous mixture with an ionic liquid and a metal-chelating group, thereby forming an ionic liquid mixture comprising the ionic liquid, the metal-chelating group, and the plurality of metal cations; and

applying an electrical potential to the ionic liquid mixture, thereby removing from the ionic liquid mixture the plurality of metal cations.

In another aspect, the disclosure provides an ionic liquid complex, comprising an ionic liquid comprising a metal-chelating group chelated to a metal cation.

In another aspect, the disclosure provides an ionic liquid mixture comprising an ionic liquid and a metal-chelating group.

### BRIEF DESCRIPTION OF THE DRAWINGS

FIG. 1A shows exemplary cationic components of ionic liquids.

FIG. 1B shows exemplary ionic liquids.

FIG. 2 shows breakthrough profiles of an exemplary ionic liquid [eth-hex-en][Tf₂N] for copper extraction at different initial copper concentrations.

FIG. 3A shows removal of Cu(NO₃)₂ from a 0.1 M aqueous solution using an exemplary ionic liquid, [eth-hex-en][Tf₂N].

FIG. 3B shows removal of Cu(NO₃)₂ from aqueous solutions of different starting concentrations using an exemplary ionic liquid, [eth-hex-en][Tf₂N].

FIG. 4 shows breakthrough profiles of an exemplary ionic liquid, [eth-hex-en]-[Tf₂N], in the extraction process for each of six metals (e.g., Ni, Ca, Pb, Al, and Mg).

FIG. 5 shows an exemplary ionic liquid, [eth-hex-en][Tf₂N], after 1^{st} cycle of chemical regeneration using HNO₃ (4 left tubes) and HCl (4 right tubes) (metals: Ag, Co, Cu, Ni).

FIG. 6 shows an exemplary ionic liquid, [eth-hex-tmeda][Tf₂N], after 1^{st} cycle of chemical regeneration using HNO₃ (4 left tubes) and HCl (4 right tubes) (metals: Ag, Co, Cu, Ni).

FIG. 7 shows, using an exemplary ionic liquid, the copper concentration in the (i) aqueous phase before extraction, (ii) organic phase after extraction, and (iii) recycled acidic aqueous solution.

FIG. 8 shows vertical H-cell for electroplating of copper directly from a saturated exemplary ionic liquid, [eth-hex-en][Tf₂N] (lower phase).

FIG. 9 shows carbon electrode after 2 cycles of silver electroplating from an exemplary ionic liquid, [eth-hex-en][Tf₂N].

FIG. 10 shows cyclic voltammetry results for an exemplary IL, [eth-hex-en][Tf₂N], loaded with a different metals.

FIG. 11 shows cyclic voltammetry results for an exemplary IL, [eth-hex-tmeda]-[Tf₂N], before and after direct electroplating of chelated silver.

FIG. 12 shows voltammograms of copper and lead in an exemplary IL, [eth-hex-en][Tf₂N]. The inset shows the independent electroplating of each metal.

FIG. 13 shows stainless steel electrode used in electroplating of lead and copper. Ion beam etching of the electrode allows the verification of electroplating. The EDS analysis of the dashed area shows the electroplating of the surface.

FIG. 14 shows a schematic of a mixer settler that achieves separation of metals from aqueous phase using ILs followed by a sequence of electroplating cells.

FIG. 15 shows a continuous flow system for copper extraction, using an exemplary IL, [eth-hex-en][Tf2N], before the extraction process.

FIG. 16 shows a continuous flow system for copper extraction, using an exemplary IL, [eth-hex-en][Tf2N], 30 minutes into the extraction process.

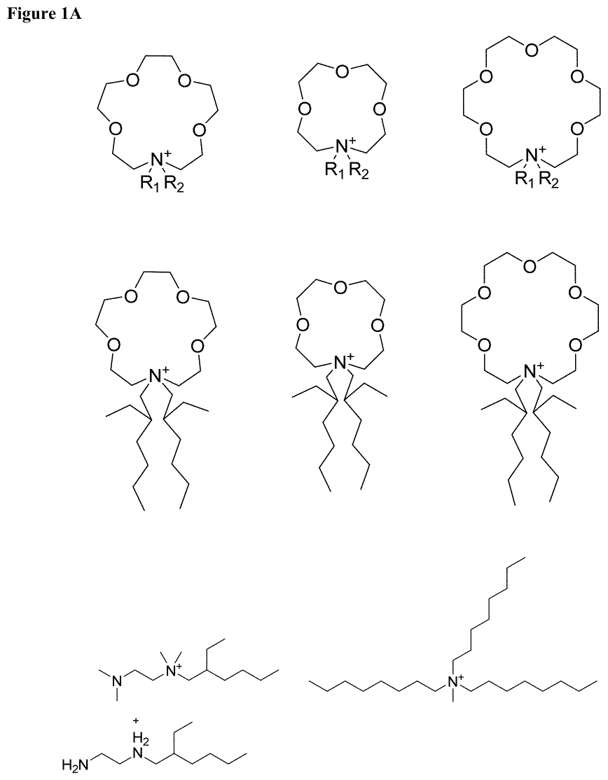

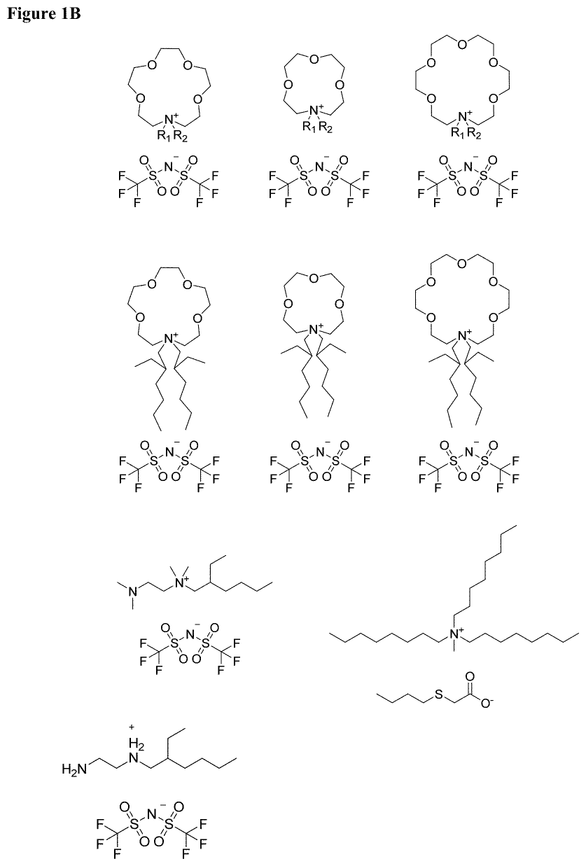

### DETAILED DESCRIPTION

This disclosure includes methods of extracting metal ions from aqueous solutions using ionic liquids (ILs), IL complexes, and mixtures comprising an IL and a metal-chelating group that can chelate to metal ions. In some embodiments, the hydrophobic task specific ILs chelate metal ions and partition them away from the aqueous phase. In some embodiments, the metal ions may then be removed from the IL by applying an electrochemical potential. This results either in electroplating or precipitation of the metal depending on the setup of the electrochemical cell.

In some embodiments, the ionic liquids described have a controlled hydrophobic-hydrophilic balance that allows them to dissolve heavy metals at relatively high concentrations (for instance, about 0.20 mol kg⁻¹). In some embodiments, the metal ions are chelated in the ion-pair region of the IL.

In some embodiments of the methods disclosed herein, the ionic liquid comprises an anion and a cation. In some embodiments, the IL does not comprise an alkylethylenediaminium cation.

In some embodiments of the methods disclosed herein, the ionic liquid comprises a metal-chelating group [22]. In some embodiments, the IL comprises a metal-chelating group, provided that the IL does not comprise a neutral ethylenediamine compound or moiety. In some embodiments, the cation of the IL comprises a metal-chelating group. In some embodiments, an IL comprising a cation comprising a metal-chelating group is referred to as a task-specific ionic liquid. In some embodiments, the cation of the IL comprises a metal-chelating group, provided that the IL does not comprise an alkylethylenediaminium cation.

In some embodiments of the methods disclosed herein, a mixture comprising an IL and a metal-chelating group is used.

In some embodiments of the methods disclosed herein, the metal-chelating group is selected from the group consisting of an ethylaminediacetic acid moiety [23], a crown ether [24, 25], a dithizone [26], a hydroxyquinoline [26], 2-thenoyltrifluoroacetone [27], a thiosalicylate [28], a salicylate [29], a thiocarbamate, a dithiocarbamate [30], an alkanolamine [31], a thioglycolate [32], an aza-crown ether [33], and a thia-crown ether [34].

### Methods of Use

Disclosed are methods of extracting metal ions using ionic liquids (ILs), IL complexes, and mixtures comprising an IL and a metal-chelating group.

In some embodiments, of the methods to extract metal ions from aqueous solution disclosed herein are for water treatment.

### Ionic Liquids Comprising a Metal-Chelating Group

In some embodiments, the IL comprises a metal-chelating group. In some embodiments, the metal-chelating group is selected from the group consisting of an ethylaminediacetic acid moiety, a crown ether, a dithizone, a hydroxyquinoline, 2-thenoyltrifluoroacetone, a thiosalicylate, a salicylate, a thiocarbamate, a dithiocarbamate, an alkanolamine, a thioglycolate, an aza-crown ether, and a thia-crown ether.

In some embodiments, the IL does not comprise an alkylethylenediaminium cation.

In some embodiments of the methods disclosed herein, metal removal from the ionic liquid mixture occurs by partitioning. In some embodiments, metal removal does not require applying an electrical potential.

In one aspect, provided herein is a method of removing metal cations from an ionic liquid mixture, comprising:

providing an ionic liquid mixture comprising an ionic liquid, wherein the IL comprises a metal-chelating group, and a plurality of metal cations,

thereby removing from the ionic liquid mixture the plurality of metal cations.

In another aspect, provided herein is a method of removing metal cations from an aqueous mixture, comprising:

providing an aqueous mixture comprising water and a plurality of metal cations;

contacting the aqueous mixture with an ionic liquid, wherein the IL comprises a metal-chelating group, thereby forming an ionic liquid mixture comprising the ionic liquid and the plurality of metal cations,

thereby removing from the ionic liquid mixture the plurality of metal cations.

In some embodiments of the methods disclosed herein, the IL comprises a metal-chelating group and the metal-chelating group is not partitioned to the aqueous mixture.

In some embodiments of the methods disclosed herein, applying the electrical potential causes the plurality of metal cations to be electrochemically reduced. In some embodiments, applying the electrical potential causes the plurality of metal cations to be electrochemically reduced to metal atoms. In some embodiments, the metal ions may be removed, and the IL regenerated, by applying an electrochemical potential. In some embodiments, the metal ion removal by applying an electrochemical potential results in electroplating. In some embodiments, the metal ion removal by applying an electrochemical potential results in precipitation of the metal. In some embodiments, the IL regeneration is an electrochemical regeneration with an oxygen evolution reaction.

In some embodiments, the metal ion removal by applying an electrochemical potential is continuous. In some embodiments, the IL regeneration by applying an electrochemical potential is continuous. In some embodiments, the metal ion removal and the IL regeneration by applying an electrochemical potential are continuous.

In some embodiments, the metal ion removal is by solvent-extraction. In some embodiments, the metal ion removal is by solvent-extraction and by stripping processes. In some embodiments, the metal ion removal is by a liquid-liquid extraction. In some embodiments, the chemical metal ion removal process is continuous. In some embodiments, the IL regeneration is by an acid wash.

In some embodiments, the selective desorption of metals is possible by controlling the electrochemical potential applied. In some embodiments, the selective desorption of a metal is selected from the metals disclosed below. In some embodiments, the methods disclosed herein result in selective desorption of a transition metal. In some embodiments, the methods disclosed herein result in selective desorption of Cu. In some embodiments, the methods disclosed herein result in selective desorption of Pb. In some embodiments, the selective desorption of a metal occurs when an electrochemical potential of 1V is applied.

In another aspect, provided herein is a method of removing metal cations from an ionic liquid mixture, comprising:

providing an ionic liquid mixture comprising an ionic liquid, wherein the IL comprises a metal-chelating group, and a plurality of metal cations; and

applying an electrical potential to the ionic liquid mixture, thereby removing from the ionic liquid mixture the plurality of metal cations.

In another aspect, provided herein is a method of removing metal cations from an aqueous mixture, comprising:

providing an aqueous mixture comprising water and a plurality of metal cations;

contacting the aqueous mixture with an ionic liquid, wherein the IL comprises a metal-chelating group, thereby forming an ionic liquid mixture comprising the ionic liquid and the plurality of metal cations; and

applying an electrical potential to the ionic liquid mixture, thereby removing from the ionic liquid mixture the plurality of metal cations.

### Ionic Liquid Mixture Comprising an Ionic Liquid and a Metal-Chelating Group

In some embodiments of the methods disclosed herein, the ionic liquid mixture comprises an ionic liquid and a metal-chelating group.

In another aspect, provided herein is a method of removing metal cations from an ionic liquid mixture, comprising:

providing an ionic liquid mixture comprising an ionic liquid, a metal-chelating group, and a plurality of metal cations; and

applying an electrical potential to the ionic liquid mixture, thereby removing from the ionic liquid mixture the plurality of metal cations.

In another aspect, provided herein is a method of removing metal cations from an aqueous mixture, comprising:

providing an aqueous mixture comprising water and a plurality of metal cations;

contacting the aqueous mixture with an ionic liquid and a metal-chelating group, thereby forming an ionic liquid mixture comprising the ionic liquid, the metal-chelating group, and the plurality of metal cations; and

applying an electrical potential to the ionic liquid mixture, thereby removing from the ionic liquid mixture the plurality of metal cations.

In some embodiments, the metal ions may be removed, and the IL regenerated, by a chemical process. In some embodiments, the IL regeneration by a chemical process is continuous. In some embodiments, the IL regeneration is by acid regeneration.

In some embodiments, the IL regeneration is by acid regeneration, and the concentration of metal cation in the acidic layer increases by at least 10-fold, at least 25-fold, at least 50-fold, at least 75-fold, at least 100-fold, at least 250-fold, at least 500-fold, at least 750-fold, at least 1,000-fold, and at least 1,500-fold. In some embodiments, the concentration of metal cation in the acidic layer increases by an amount selected from the group consisting of about 10-fold, about 20-fold, about 25-fold, about 30-fold, about 40-fold, about 50-fold, about 60-fold, about 70-fold, about 75-fold, about 80-fold, about 90-fold, about 100-fold, about 110-fold, about 120-fold, about 125-fold, about 130-fold, about 140-fold, about 50-fold, about 60-fold, about 70-fold, about 75-fold, about 80-fold, about 90-fold, about 100-fold, about 110-fold, about 120-fold, about 125-fold, about 130-fold, about 140-fold, about 150-fold, about 160-fold, about 170-fold, about 175-fold, about 180-fold, about 190-fold, about 200-fold, about 210-fold, about 220-fold, about 225-fold, about 230-fold, about 240-fold, about 250-fold, about 260-fold, about 270-fold, about 275-fold, about 280-fold, about 290-fold, about 300-fold, about 310-fold, about 320-fold, about 325-fold, about 330-fold, about 340-fold, about 350-fold, about 360-fold, about 370-fold, about 375-fold, about 380-fold, about 390-fold, about 400-fold, about 410-fold, about 420-fold, about 425-fold, about 430-fold, about 440-fold, about 450-fold, about 460-fold, about 470-fold, about 475-fold, about 480-fold, about 490-fold, about 500-fold, about 510-fold, about 520-fold, about 525-fold, about 530-fold, about 540-fold, about 550-fold, about 560-fold, about 570-fold, about 575-fold, about 580-fold, about 590-fold, about 600-fold, about 610-fold, about 620-fold, about 625-fold, about 630-fold, about 640-fold, about 650-fold, about 660-fold, about 670-fold, about 675-fold, about 680-fold, about 690-fold, about 700-fold, about 710-fold, about 720-fold, about 725-fold, about 730-fold, about 740-fold, about 750-fold, about 760-fold, about 770-fold, about 775-fold, about 780-fold, about 790-fold, about 800-fold, about 810-fold, about 820-fold, about 825-fold, about 830-fold, about 840-fold, about 850-fold, about 860-fold, about 870-fold, about 875-fold, about 880-fold, about 890-fold, about 900-fold, about 910-fold, about 920-fold, about 925-fold, about 930-fold, about 940-fold, about 950-fold, about 960-fold, about 970-fold, about 975-fold, about 980-fold, about 990-fold, and about 1,000-fold.

In some embodiments of the methods disclosed herein, the metal-chelating group is an ethylaminediacetic acid moiety.

In some embodiments of the methods disclosed herein, the metal-chelating group is a crown ether.

In some embodiments of the methods disclosed herein, the metal-chelating group is a dithizone.

In some embodiments of the methods disclosed herein, the metal-chelating group is a hydroxyquinoline.

In some embodiments of the methods disclosed herein, the metal-chelating group is 2-thenoyltrifluoroacetone.

In some embodiments of the methods disclosed herein, the metal-chelating group is a thiosalicylate.

In some embodiments of the methods disclosed herein, the metal-chelating group is a salicylate.

In some embodiments of the methods disclosed herein, the metal-chelating group is a thiocarbamate or a dithiocarbamate.

In some embodiments of the methods disclosed herein, the metal-chelating group is an alkanolamine.

In some embodiments of the methods disclosed herein, the metal-chelating group is a thioglycolate.

In some embodiments of the methods disclosed herein, the metal-chelating group is an aza-crown ether.

In some embodiments of the methods disclosed herein, the metal-chelating group is a thia-crown ether.

In some embodiments of the methods disclosed herein, the ionic liquid comprises a cation and an anion; and the cation is represented by the following structural formula I:

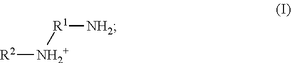

wherein, independently for each occurrence:

R¹ is —(C(R)₂)ₙ—;

n is 2, or 3;

R² is —(C(R′)₂)ₘ-R″;

m is 1, 2, 3, 4, 5, 6, 7, 8, 9, or 10; and

R is H, F, C₁-C₃ alkyl, or C₁-C₃ fluoroalkyl;

R′ is H, F, C₁-C₈ alkyl, or C₁-C₈ fluoroalkyl; and

R″ is H, F, C₁-C₃ alkyl, C₁-C₃ fluoroalkyl, C₁-C₃ alkyloxy, C₁-C₃ fluoroalkyloxy, C₆-C₁₀ aryl, C₂-C₈ alkenyl or C₂-C₈ fluoroalkenyl; wherein each instance of C₆-C₁₀ aryl is optionally substituted with one, two, three, four or five substituents independently selected from the group consisting of F, C₁-C₃ alkyl, C₁-C₃ fluoroalkyl, C₁-C₃ alkyloxy, and C₁-C₃ fluoroalkyloxy.

The variables in formula I may be further selected as described below.

In some embodiments of the methods disclosed herein, the ionic liquid comprises a cation and an anion; and the cation is represented by the following structural formula II:

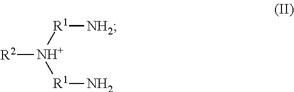

wherein, independently for each occurrence:

R¹ is —(C(R)₂)ₙ—;

n is 2, or 3;

R² is —(C(R′)₂)ₘ-R″;

m is 1, 2, 3, 4, 5, 6, 7, 8, 9, or 10; and

R is H, F, C₁-C₃ alkyl, or C₁-C₃ fluoroalkyl;

R′ is H, F, C₁-C₈ alkyl, or C₁-C₈ fluoroalkyl; and

R″ is H, F, C₁-C₃ alkyl, C₁-C₃ fluoroalkyl, C₁-C₃ alkyloxy, C₁-C₃ fluoroalkyloxy, C₆-C₁₀ aryl, C₂-C₈ alkenyl or C₂-C₈ fluoroalkenyl; wherein each instance of C₆-C₁₀ aryl is optionally substituted with one, two, three, four or five substituents independently selected from the group consisting of F, C₁-C₃ alkyl, C₁-C₃ fluoroalkyl, C₁-C₃ alkyloxy, and C₁-C₃ fluoroalkyloxy.

The variables in formula II may be further selected as described below.

In some embodiments of the ionic liquids disclosed herein, the anion is boron tetrafluoride (BF₄⁻), phosphorus tetrafluoride, phosphorus hexafluoride (PF₆⁻), alkylsulfonate, fluoroalkylsulfonate, arylsulfonate, bis(alkylsulfonyl)amide, bis(fluoroalkylsulfonyl)amide, bis(fluoroalkylsulfonyl)imide, bis(arylsulfonyl)amide, (fluoroalkylsulfonyl)-(fluoroalkylcarbonyl)amide, (fluoroalkylsulfonyl)-(fluoroalkylcarbonyl)imide, halide, nitrate, nitrite, sulfate, hydrogensulfate, alkyl sulfate, aryl sulfate, carbonate, bicarbonate, carboxylate, phosphate, hydrogen phosphate, dihydrogen phosphate, hypochlorite, an anionic site of a cation-exchange resin, an acetate, a bicarbonate, a carbonate, a halide, a nitrate, nonaflate, a sulfate, a sulfonate, a phosphate, a triflate. In some embodiments, the anion is boron tetrafluoride, phosphorus tetrafluoride, phosphorus hexafluoride, alkylsulfonate, fluoroalkylsulfonate, arylsulfonate, bis(alkylsulfonyl)amide, bis(fluoroalkylsulfonyl)amide, bis(arylsulfonyl)amide, (fluoroalkylsulfonyl)(fluoroalkylcarbonyl)amide, halide, nitrate, nitrite, sulfate, hydrogensulfate, alkyl sulfate, aryl sulfate, carbonate, bicarbonate, carboxylate, phosphate, hydrogen phosphate, dihydrogen phosphate, hypochlorite, or an anionic site of a cation-exchange resin. In some embodiments, the anion is boron tetrafluoride, phosphorus tetrafluoride, phosphorus hexafluoride, halide, nitrate, nitrite, sulfate, hydrogensulfate, carbonate, bicarbonate, phosphate, hydrogen phosphate, dihydrogen phosphate, hypochlorite, or an anionic site of a cation-exchange resin. In some embodiments, the anion is C₁-C₁₂ alkylsulfonate, C₁-C₁₂ fluoroalkylsulfonate, C₆-C₁₀ arylsulfonate, C₂-C₂₄ bis(alkylsulfonyl)amide, C₂-C₂₄ bis(fluoroalkylsulfonyl)amide, C₁₂-C₂₀ bis(arylsulfonyl)amide, C₂-C₂₄ (fluoroalkylsulfonyl)(fluoroalkylcarbonyl)amide, C₁-C₁₂ alkyl sulfate, C₆-C₁₀ aryl sulfate, or C₁-C₁₂ carboxylate. In some embodiments, the anion is boron tetrafluoride (BF₄⁻), phosphorus hexafluoride (PF₆⁻), methanesulfonate, trifluoromethanesulfonate, benzenesulfonate, p-toluenesulfonate, bis(methanesulfonyl)amide, bis(trifluoromethanesulfonyl)amide, bis(trifluoromethanesulfonyl)imide, bis(benzenesulfonyl)amide, or bis(p-toluenesulfonyl)amide. In some embodiments, the anion is methanesulfonate, trifluoromethanesulfonate, benzenesulfonate, p-toluenesulfonate, bis(methanesulfonyl)amide, bis(trifluoromethanesulfonyl)amide, bis(benzenesulfonyl)amide, or bis(p-toluenesulfonyl)amide. In some embodiments, the anion is bis(methanesulfonyl)amide, bis(trifluoromethanesulfonyl)amide, bis(benzenesulfonyl)amide, or bis(p-toluenesulfonyl)amide. In some embodiments, the anion is boron tetrafluoride (BF₄⁻), phosphorus hexafluoride (PF₆⁻), methanesulfonate, trifluoromethanesulfonate, benzenesulfonate, p-toluenesulfonate, bis(methanesulfonyl)amide, bis(trifluoromethanesulfonyl)amide, bis(benzenesulfonyl)amide, or bis(p-toluenesulfonyl)amide. In some embodiments, the anion is methanesulfonate, trifluoromethanesulfonate, benzenesulfonate, p-toluenesulfonate, bis(methanesulfonyl)amide, bis(trifluoromethanesulfonyl)amide, bis(benzenesulfonyl)amide, or bis(p-toluenesulfonyl)amide. In some embodiments, the anion is bis(methanesulfonyl)amide, bis(trifluoromethanesulfonyl)amide, bis(benzenesulfonyl)amide, or bis(p-toluenesulfonyl)amide. In some embodiments, the anion is bis(trifluoromethanesulfonyl)amide or (trifluoromethanesulfonyl)(trifluoroacetyl)amide. In some embodiments, the anion is bis(trifluoroethanesulfonyl)amide. In some embodiments, the anion is bis(trifluoromethanesulfonyl)imide.

In some embodiments of the ionic liquids disclosed herein, the anion is antimicrobial. For example, the antimicrobial anion is penicillin or a related carboxylic acid (e.g., ampicillin, carbenicillin, oxacillin, narcillin, and cloxacillin). In some embodiments, the antimicrobial anion is ampicillin.

In some embodiments of the ionic liquids disclosed herein, the cation is an ammonium, an imidazolium, an oxazolium, a pyrazinium, a pyridazinium, a pyrazolium, a pyridinium, a pyrimidinium, a sulfonium, a thiazolium, or a triazolium.

In some embodiments, the ionic liquid comprises poly(diallyldimethylammonium) cations and ampicillin counterions.

In some embodiments of the methods disclosed herein, the metal cation has a charge of +1. In some embodiments, the metal cation is a cation of Ag.

In some embodiments of the methods disclosed herein, the metal cation has a charge of +2. In some embodiments, the metal cation is a cation of Ca, Cd, Co, Cr, Cu, Er, Fe, Hg, Mg, Mn, Nb, Ni, Pb, Pd, Sc, Sn, Sr, V, or Zn. In some embodiments, the metal cation is a cation of Mg, Fe, Hg, Sr, Sn, Ca, Cd, Zn, Co, Cu, Pb, Ni, Sc, V, Cr, or Mn. In some embodiments, the metal cation is a cation of Ni, Zn, Cu, Pb, or Co. In some embodiments, the metal cation is a cation of Ca, Cu, or Zn. In some embodiments, the metal cation is a cation of Cu. In some embodiments, the metal cation is a cation of Fe, Ni, Zn, Co, Sc, V, Cr, or Mn. In some embodiments, the metal cation is a cation of Ni, Zn, Co, Sc, V, Cr, or Mn. In some embodiments, the metal cation is a cation of Pd, Nb, Hg, and Er. In some embodiments, the metal cation is a polycation of Hg (i.e., Hg₂, Hg₃, or Hg₄).

In some embodiments of the methods disclosed herein, the metal cation has a charge of +3. In some embodiments, the metal cation is a cation of Ce, Dy, Er, Eu, Fe, Gd, Ho, La, Lu, Nb, Nd, Pm, Pr, Sm, Tb, Tm, or Yb. In some embodiments, the metal cation is a cation of Fe, La, Ce, Pr, Nd, Pm, Sm, Eu, Gd, Tb, Dy, Ho, Er, Tm, Yb, or Lu. In some embodiments, the metal cation is a cation of Ac, Th, Pa, U, Np, Pu, Am, Cm, Bk, Cf, Es, Fm, Md, No, or Lr. In some embodiments, the metal cation is a cation of Fe.

In some embodiments of the methods disclosed herein, an ionic liquid complex comprising an ionic liquid chelated to a metal cation is formed. In some embodiments, an ionic liquid complex comprising an ionic liquid comprising a metal-chelating group chelated to a metal cation is formed.

In some embodiments of the methods and complexes disclosed herein, the ionic liquid mixture further comprises water.

In some embodiments of the methods and complexes disclosed herein, the ionic liquid mixture further comprises an oil. In some embodiments, the ionic liquid further comprises an oil. For example, an oil includes a hydrophobic oil, a heavy oil, a vacuum pump oil, a silicon oil, a fluorinated oil, an oil mixture comprising a chelating moiety (e.g., a crown ether or a cyclam), coconut oil, corn oil, cottonseed oil, fish oil, grape seed oil, hazelnut oil, a hydrogenated vegetable oil, olive oil, palm seed oil, peanut oil, safflower oil, sesame oil, soybean oil, sunflower oil, walnut oil, etc., and any combination thereof.

In some embodiments of the methods and complexes disclosed herein, the ionic liquid further comprises an organic solvent (e.g., benzene, benzyl benzoate, chlorobenzene, chloroform, cyclohexane, decane, dichloromethane, diethylether, ethyl acetate, gasoline, naptha, naphthalene, n-hexane, n-heptane, n-decanol, isododecane, n-dodecane, di-2-ethylhexylphosphoric acid, pentane, tibutylphosphate, toluene, triethylamine, xylene, and kerosene, etc., and any combination thereof).

In some embodiments, the ionic liquid further comprises one or more of water, oil, and an organic solvent.

In some embodiments, the IL has a wide electrochemical window. The electrochemical window is the voltage range where the IL is neither oxidized nor reduced. In some embodiments, the potential range for which the IL is stable. In some embodiments, the IL is stable from about 0.5 EV to about 9 EV. In some embodiments, the IL is stable at about 0.5 EV, about 0.6 EV, about 0.7 EV, about 0.8 EV, about 0.9 EV, about 1.0 EV, about 1.1 EV, about 1.2 EV, about 1.3 EV, about 1.4 EV, about 1.5 EV, about 1.6 EV, about 1.7 EV, about 1.8 EV, about 1.9 EV, about 2.0 EV, about 2.1 EV, about 2.2 EV, about 2.3 EV, about 2.4 EV, about 2.5 EV, about 2.6 EV, about 2.7 EV, about 2.8 EV, about 2.9 EV, about 3.0 EV, about 3.1 EV, about 3.2 EV, about 3.3 EV, about 3.4 EV, about 3.5 EV, about 3.6 EV, about 3.7 EV, about 3.8 EV, about 3.9 EV, about 4.0 EV, about 4.1 EV, about 4.2 EV, about 4.3 EV, about 4.4 EV, about 4.5 EV, about 4.6 EV, about 4.7 EV, about 4.8 EV, about 4.9 EV, about 5.0 EV, about 5.1 EV, about 5.2 EV, about 5.3 EV, about 5.4 EV, about 5.5 EV, about 5.6 EV, about 5.7 EV, about 5.8 EV, about 5.9 EV, about 6.0 EV, about 6.1 EV, about 6.2 EV, about 6.3 EV, about 6.4 EV, about 6.5 EV, about 6.6 EV, about 6.7 EV, about 6.8 EV, about 6.9 EV, about 7.0 EV, about 7.1 EV, about 7.2 EV, about 7.3 EV, about 7.4 EV, about 7.5 EV, about 7.6 EV, about 7.7 EV, about 7.8 EV, about 7.9 EV, about 8.0 EV, about 8.1 EV, about 8.2 EV, about 8.3 EV, about 8.4 EV, about 8.5 EV, about 8.6 EV, about 8.7 EV, about 8.8 EV, about 8.9 EV, and about 9.0 EV. In some embodiments, the IL is stable from about 0.5 EV to about 7 EV. In some embodiments, the IL is stable from about 2 EV to about 7 EV. In some embodiments, the IL is stable from about 3 EV to about 5 EV. In some embodiments, the IL is stable from about 4 EV to about 5 EV.

In some embodiments, the IL is stable over a range of about 9 EV. In some embodiments, the IL is stable over a range of about 1 EV to about 9 EV. In some embodiments, the IL is stable over a range of about 1.0 EV, about 1.1 EV, about 1.2 EV, about 1.3 EV, about 1.4 EV, about 1.5 EV, about 1.6 EV, about 1.7 EV, about 1.8 EV, about 1.9 EV, about 2.0 EV, about 2.1 EV, about 2.2 EV, about 2.3 EV, about 2.4 EV, about 2.5 EV, about 2.6 EV, about 2.7 EV, about 2.8 EV, about 2.9 EV, about 3.0 EV, about 3.1 EV, about 3.2 EV, about 3.3 EV, about 3.4 EV, about 3.5 EV, about 3.6 EV, about 3.7 EV, about 3.8 EV, about 3.9 EV, about 4.0 EV, about 4.1 EV, about 4.2 EV, about 4.3 EV, about 4.4 EV, about 4.5 EV, about 4.6 EV, about 4.7 EV, about 4.8 EV, about 4.9 EV, about 5.0 EV, about 5.1 EV, about 5.2 EV, about 5.3 EV, about 5.4 EV, about 5.5 EV, about 5.6 EV, about 5.7 EV, about 5.8 EV, about 5.9 EV, about 6.0 EV, about 6.1 EV, about 6.2 EV, about 6.3 EV, about 6.4 EV, about 6.5 EV, about 6.6 EV, about 6.7 EV, about 6.8 EV, about 6.9 EV, about 7.0 EV, about 7.1 EV, about 7.2 EV, about 7.3 EV, about 7.4 EV, about 7.5 EV, about 7.6 EV, about 7.7 EV, about 7.8 EV, about 7.9 EV, about 8.0 EV, about 8.1 EV, about 8.2 EV, about 8.3 EV, about 8.4 EV, about 8.5 EV, about 8.6 EV, about 8.7 EV, about 8.8 EV, about 8.9 EV, and about 9.0 EV. In some embodiments, the IL is stable over a range of about 7 EV. In some embodiments, the IL is stable over a range of about 6 EV. In some embodiments, the IL is stable over a range of about 5 EV. In some embodiments, the IL is stable over a range of about 4 EV. In some embodiments, the IL is stable over a range of about 2 EV. In some embodiments, the IL is stable over a range greater than about 1.5 EV. The electrochemical window for water is about 1.23 EV.

In some embodiments, the electrochemistry kinetics are fast.

In some embodiments, the ILs are recyclable. In some embodiments, the ILs are partially recyclable. In some embodiments, the ILs are fully recyclable. In some embodiments, at least 75%, at least 80%, at least 85%, at least 90%, at least 95%, at least 96%, at least 97%, at least 98%, or at least 99% of the ILs are recycled after applying an electrochemical potential. In some embodiments, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.5%, about 99.9%, or about 100% of the ILs are recycled after applying an electrochemical potential.

In some embodiments of the methods disclosed herein, the ionic liquid comprises hydrophobic moieties. In some embodiments, the hydrophobic task specific ILs chelate metal ions. In some embodiments, the hydrophobic ILs are water-immiscible. In some embodiments, the hydrophobic ILs have low miscibility with water.

The physicochemical properties (e.g., viscosity) of the ionic liquids disclosed herein can be adjusted based on the chemical structure. In some embodiments of the methods disclosed herein, the ionic liquid has a low viscosity so as not to impede flow of a sample through the system.

In some embodiments of the methods disclosed herein, metal has a diffusion coefficient in ionic liquids of less than about 1×10⁻⁵ m²/s. In some embodiments, the metal has a diffusion coefficient in ILs of less than about 1×10⁻⁷ m²/s. In some embodiments, the metal has a diffusion coefficient in ILs of less than about 1×10⁻⁹ m²/s. In some embodiments, the metal has a diffusion coefficient in ILs from about 1×10⁻²⁰ m²/s to about 1×10⁻⁵ m²/s. In some embodiments, the metal has a diffusion coefficient in ILs selected from the group consisting of about 1×10⁻²⁰ m²/s, about 1×10⁻¹⁹ m²/s, about 1×10⁻¹⁸ m²/s, about 1×10⁻¹⁷ m²/s, about 2×10⁻¹⁷ m²/s, about 3×10⁻¹⁷ m²/s, about 4×10⁻¹⁷ m²/s, about 5×10⁻¹⁷ m²/s, about 6×10⁻¹⁷ m²/s, about 7×10⁻¹⁷ m²/s, about 8×10⁻¹⁷ m²/s, about 9×10⁻¹⁷ m²/s, about 1×10⁻¹⁶ m²/s, about 2×10⁻¹⁶ m²/s, about 3×10⁻¹⁶ m²/s, about 4×10⁻¹⁶ m²/s, about 5×10⁻¹⁶ m²/s, about 6×10⁻¹⁶ m²/s, about 7×10⁻¹⁶ m²/s, about 8×10⁻¹⁶ m²/s, about 9×10⁻¹⁶ m²/s, about 1×10⁻¹⁵ m²/s, about 2×10⁻¹⁵ m²/s, about 3×10⁻¹⁵ m²/s, about 4×10⁻¹⁵ m²/s, about 5×10⁻¹⁵ m²/s, about 6×10⁻¹⁵ m²/s, about 7×10⁻¹⁵ m²/s, about 8×10⁻¹⁵ m²/s, about 9×10⁻¹⁵ m²/s, about 1×10⁻¹⁴ m²/s, about 2×10⁻¹⁴ m²/s, about 3×10⁻¹⁴ m²/s, about 4×10⁻¹⁴ m²/s, about 5×10⁻¹⁴ m²/s, about 6×10⁻¹⁴ m²/s, about 7×10⁻¹⁴ m²/s, about 8×10⁻¹⁴ m²/s, about 9×10⁻¹⁴ m²/s, about 1×10⁻¹³ m²/s, about 2×10⁻¹³ m²/s, about 3×10⁻¹³ m²/s, about 4×10⁻¹³ m²/s, about 5×10⁻¹³ m²/s, about 6×10⁻¹³ m²/s, about 7×10⁻¹³ m²/s, about 8×10⁻¹³ m²/s, about 9×10⁻¹³ m²/s, about 1×10⁻¹² m²/s, about 2×10⁻¹² m²/s, about 3×10⁻¹² m²/s, about 4×10⁻¹² m²/s, about 5×10⁻¹² m²/s, about 6×10⁻¹² m²/s, about 7×10⁻¹² m²/s, about 8×10⁻¹² m²/s, about 9×10⁻¹² m²/s, about 1×10⁻¹¹ m²/s, about 2×10⁻¹¹ m²/s, about 3×10⁻¹¹ m²/s, about 4×10⁻¹¹ m²/s, about 5×10⁻¹¹ m²/s, about 6×10⁻¹¹ m²/s, about 7×10⁻¹¹ m²/s, about 8×10⁻¹¹ m²/s, about 9×10⁻¹¹ m²/s, about 1×10⁻¹⁰ m²/s, about 2×10⁻¹⁰ m²/s, about 3×10⁻¹⁰ m²/s, about 4×10⁻¹⁰ m²/s, about 5×10⁻¹⁰ m²/s, about 6×10⁻¹⁰ m²/s, about 7×10⁻¹⁰ m²/s, about 8×10⁻¹⁰ m²/s about 9×10⁻¹⁰ m²/s, about 1×10⁻⁹ m²/s, about 2×10⁻⁹ m²/s, about 3×10⁻⁹ m²/s, about 4×10⁻⁹ m²/s, about 5×10⁻⁹ m²/s, about 6×10⁻⁹ m²/s, about 7×10⁻⁹ m²/s, about 8×10⁻⁹ m²/s, about 9×10⁻⁹ m²/s, about 1×10⁻⁸ m²/s, about 2×10⁻⁸ m²/s, about 3×10⁻⁸ m²/s, about 4×10⁻⁸ m²/s, about 5×10⁻⁸ m²/s, about 6×10⁻⁸ m²/s, about 7×10⁻⁸ m²/s, about 8×10⁻⁸ m²/s, about 9×10⁻⁸ m²/s, about 1×10⁻⁷ m²/s, about 2×10⁻⁷ m²/s, about 3×10⁻⁷ m²/s, about 4×10⁻⁷ m²/s, about 5×10⁻⁷ m²/s, about 6×10⁻⁷ m²/s, about 7×10⁻⁷ m²/s, about 8×10⁻⁷ m²/s, about 9×10⁻⁷ m²/s, about 1×10⁻⁶ m²/s, and about 1×10⁻⁵ m²/s. In some embodiments, the metal has a diffusion coefficient in ILs from about 1×10⁻¹⁵ m²/s to about 1×10⁻⁷ m²/s. In some embodiments, the metal has a diffusion coefficient in ILs from about 1×10⁻¹³ m²/s to about 1×10⁻⁹ m²/s.

In some embodiments of the methods disclosed herein, the ionic liquid has a high selectivity for transition metal ions (e.g., Fe, Hg, Cd, Zn, Co, Cu, Ni, Sc, V, Cr, and Mn). In some embodiments, the ionic liquid has a removal efficiency of at least 75%, at least 80%, at least 85%, at least 90%, at least 95%, at least 96%, at least 97%, at least 98%, or at least 99%. In some embodiments, the ionic liquid has a removal efficiency is about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.5%, about 99.9%, or about 100%.

In some embodiments of the methods disclosed herein, when an ionic liquid complex comprising an ionic liquid chelated to a metal cation is formed, the concentration of metal cation in the IL significantly increases. In some embodiments, the concentration of metal cation in the IL increases at least 10-fold, at least 25-fold, at least 50-fold, at least 75-fold, at least 100-fold, at least 250-fold, at least 500-fold, at least 750-fold, at least 1,000-fold, at least 1,500-fold, at least 2,000-fold, at least 2,500-fold, or at least 3,000-fold. In some embodiments, the concentration of metal cation in the IL increases by an amount selected from the group consisting of about 10-fold, about 25-fold, about 50-fold, about 75-fold, about 100-fold, about 125-fold, about 150-fold, about 175-fold, about 200-fold, about 225-fold, about 250-fold, about 275-fold, about 300-fold, about 325-fold, about 350-fold, about 375-fold, about 400-fold, about 425-fold, about 450-fold, about 475-fold, about 500-fold, about 525-fold, about 550-fold, about 575-fold, about 600-fold, about 625-fold, about 650-fold, about 675-fold, about 700-fold, about 725-fold, about 750-fold, about 775-fold, about 800-fold, about 825-fold, about 850-fold, about 875-fold, about 900-fold, about 925-fold, about 950-fold, about 975-fold, about 1,000-fold, about 1,025-fold, about 1,050-fold, about 1,075-fold, about 1,100-fold, about 1,125-fold, about 1,150-fold, about 1,175-fold, about 1,200-fold, about 1,225-fold, about 1,250-fold, about 1,275-fold, about 1,300-fold, about 1,325-fold, about 1,350-fold, about 1,375-fold, about 1,400-fold, about 1,425-fold, about 1,450-fold, about 1,475-fold, about 1,500-fold, about 1,525-fold, about 1,550-fold, about 1,575-fold, about 1,600-fold, about 1,625-fold, about 1,650-fold, about 1,675-fold, about 1,700-fold, about 1,725-fold, about 1,750-fold, about 1,775-fold, about 1,800-fold, about 1,825-fold, about 1,850-fold, about 1,875-fold, about 1,900-fold, about 1,925-fold, about 1,950-fold, about 1,975-fold, about 2,000-fold, about 2,100-fold, about 2,200-fold, about 2,300-fold, about 2,400-fold, about 2,500-fold, about 2,600-fold, about 2,700-fold, about 2,800-fold, about 2,900-fold, and about 3,000-fold.

In some embodiments of the methods disclosed herein, when an ionic liquid complex comprising an ionic liquid chelated to a metal cation is formed, the ionic liquid complex does not precipitate out of an aqueous solution. This is an improvement over other known ionic liquid complexes that precipitate out of aqueous solution, which can interfere with flow of a sample through the system.

In some embodiments of the ionic liquids and ionic liquid complexes disclosed herein, the ionic liquid or the ionic liquid complex forms a microemulsion, an emulsion, or a gel.

### Compounds of the Disclosure

### Ionic Liquid Complexes

Another aspect of the disclosure relates to an ionic liquid complex, comprising an ionic liquid an ionic liquid comprising a metal-chelating group chelated to a metal cation. In some embodiments, the metal-chelating group is selected from the group consisting of an ethylaminediacetic acid moiety, a crown ether, a dithizone, a hydroxyquinoline, 2-thenoyltrifluoroacetone, a thiosalicylate, a salicylate, a thiocarbamate, a dithiocarbamate, an alkanolamine, a thioglycolate, an aza-crown ether, and a thia-crown ether.

In some embodiments, the IL comprises an ethylaminediacetic acid moiety as a metal-chelating group. In some embodiments, the IL comprises

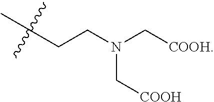 [SMILES: *CCN(CC(=O)O)CC(=O)O]

In some embodiments, the IL is

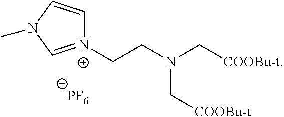

In some embodiments, the IL comprises a crown ether as a metal-chelating group. In some embodiments, the IL comprises a crown ether selected from the group consisting of 2-hydroxymethyl-12-crown-4; 12-crown-4; 15-crown-5; 2-aminomethyl-15-crown-5; 2-hydroxymethyl-15-crown-5; 4′-aminobenzo-15-crown-5; 24′-formylbenzo-15-crown-5; 4′-nitrobenzo-15-crown-5; 2,3-naphtho-15-crown-5; benzo-15-crown-5; dibenzo-15-crown-5; 18-crown-6; 2-aminomethyl-18-crown-6; benzo-18-crown-6; 2-hydroxymethyl-18-crown-6; 4′-aminobenzo-18-crown-6; dicyclohexano-18-crown-6; dibenzo-18-crown-6; 4′-aminodibenzo-18-crown-6; dibenzo-21-crown-7; dibenzo-24-crown-8; and dibenzo-30-crown-10.

In some embodiments, the IL comprises a dithizone as a metal-chelating group. In some embodiments, the IL comprises

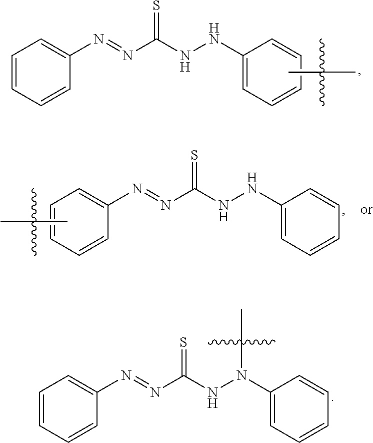

In some embodiments, the IL comprises a hydroxyquinoline as a metal-chelating group. In some embodiments, the IL comprises

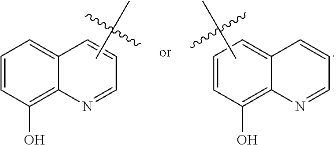

In some embodiments, the IL comprises 2-thenoyltrifluoroacetone as a metal-chelating group. In some embodiments, the IL or the IL mixture comprises

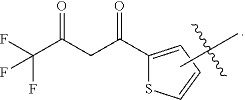

In some embodiments, the IL comprises a thiosalicylate or thiosalicylic acid as a metal-chelating group. In some embodiments, the IL comprises

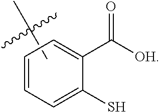

In some embodiments, the IL is trioctylammonium thiosalicylate or tricaprylmethylammonium thiosalicylate.

In some embodiments, the IL comprises a salicylate or salicylic acid as a metal-chelating group. In some embodiments, the IL or the IL mixture comprises

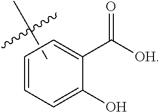

In some embodiments, the IL is trioctylammonium salicylate or tricaprylmethylammonium salicylate.

In some embodiments, the IL comprises a thiocarbamate or a dithiocarbamate as a metal-chelating group. In some embodiments, the IL comprises

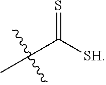 [SMILES: *C(=S)S]

In some embodiments, the IL comprises a dithiocarbamate selected from the group consisting of

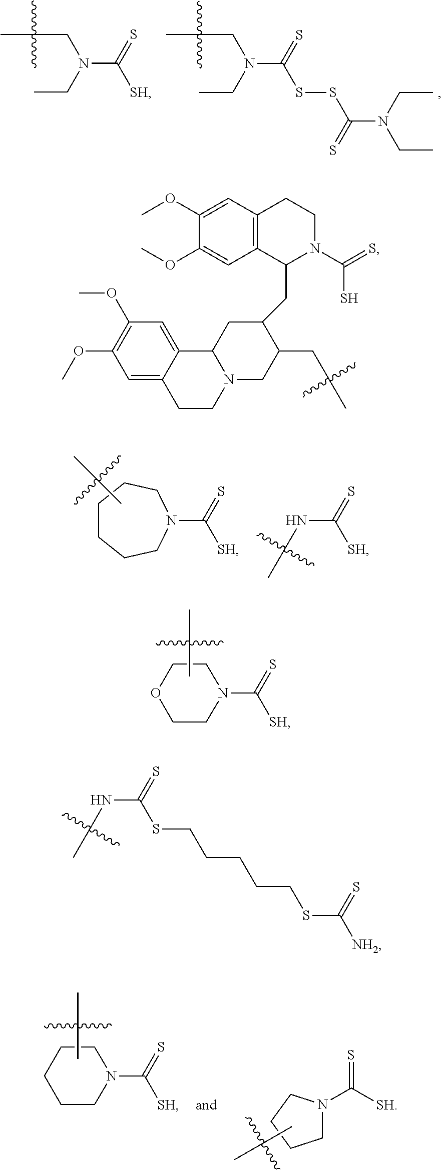

In some embodiments, the IL comprises an alkanolamine as a metal-chelating group. In some embodiments, the IL comprises an alkanolamine selected from the group consisting of

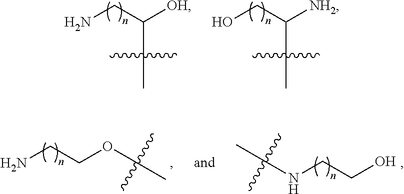

wherein n is an integer from 0 to 10. In some embodiments, the IL is selected from the group consisting of monoethanolamine bis(trifluoroethanesulfonyl)amide, diethanolamine bis(trifluoroethanesulfonyl)amide, 2-((2-aminoethyl)amino)ethan-1-ol bis(trifluoroethanesulfonyl)amide, and 2,2′-(ethane-1,2-diylbis(oxy))bis(ethan-1-amine) bis(trifluoroethanesulfonyl)amide.

In some embodiments, the IL comprises a thioglycolate as a metal-chelating group. In some embodiments, the IL comprises a thioglycolate selected from the group consisting of

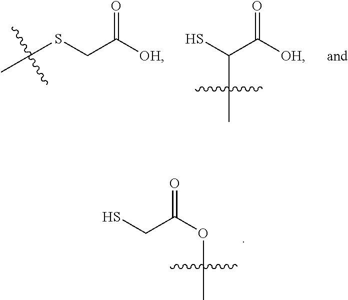 [SMILES: *SCC(=O)O ; *C(S)C(=O)O ; *OC(=O)CS]

In some embodiments, the IL is selected from the group consisting of methyltrioctylammonium butylsulfanyl acetate, methyltrioctylammonium pentylsulfanyl acetate, methyltrioctylammonium hexylsulfanyl acetate, methyltrioctylammonium benzylsulfanyl acetate, methyltrioctylphosphonium butylsulfanyl acetate, methyltrioctylphosphonium pentylsulfanyl acetate, methyltrioctylphosphonium hexylsulfanyl acetate, and methyltrioctylphosphonium benzylsulfanyl acetate.

In some embodiments, the IL comprises an aza-crown ether as a metal-chelating group. In some embodiments, the aza-crown ether is a ring containing several nitrogen atoms. In some embodiments, the IL comprises an aza-crown ether selected from the group consisting of 1-aza-12-crown-4; 1,7-diaza-12-crown-4; 4,10-diaza-12-crown-4; 1-aza-15-crown-5; 4,10-diaza-15-crown-5; N-phenylaza-15-crown-5; 1-aza-18-crown-6; 4,13-diaza-18-crown-6; 7,16-dibenzyl-1,4,10,13-tetraoxa-7,16-diazacycloocta-decane; 4,7,13,16,21-pentaoxa-1,10-diazabicyclo[8.8.5]tricosane; 4,7,13,16,21,24-hexaoxa-1,10-diazabicyclo[8.8.8]hexacosane; 5,6-benzo-4,7,13,16,21,24-hexaoxa-1,10-diazabicyclo[8.8.8]hexacos-5-ene; 5,6,14,15-dibenzo-1,4-dioxa-8,12-diazacyclopentadeca-5,14-diene; 1,4,10,13-tetraoxa-7,16-diazacyclooctadecane-1,4,7,10-tetraacetic acid; 1,4,7-triazacyclononane; 1,4,7-trimethyl-1,4,7-triazacyclononane; cyclen; hexacyclen; 1,4,7,10-tetraazacyclododecane; tri-tent-butyl 1,4,7,10-tetraazacyclododecane-1,4,7-triacetate; tri-tent-butyl 1,4,7,10-tetraazacyclododecane-1,4,7,10-tetraacetate; 1,4,8,12-tetraazacyclopentadecane; 1,4,8,11-tetraazacyclotetradecane; 1,4,8,11-tetraazacyclotetradecane-5,7-dione; and 1,4,8,11-tetramethyl-1,4,8,11-tetraazacyclotetradecane. In some embodiments, the IL comprises an aza-crown ether selected from the group consisting of 1-aza-12-crown-4; 1-aza-15-crown-5; and 1-aza-18-crown-6.

In some embodiments, the IL comprises a thia-crown ether as a metal-chelating group. In some embodiments, the IL comprises a thia-crown ether selected from the group consisting of 1,4,7-trithiacyclononane; 3,6,9-trithia-1(2,5)-thiophenacyclodecaphane; 1,4,7,10-tetrathiacyclododecane; 1,4,8,11-tetrathiacyclotetradecane; 1,4,7,10,13-pentathiacyclopentadecane; 1,5,9,13-tetrathiacyclohexadecane; 1,5,9,13-tetrathiacyclohexadecane-3,11-diol; 1,4,7,10,13,16-hexathiacyclooctadecane; 2,3,5,6,8,9,11,12,14,15-decahydrobenzo[b][1,4,7,10,13,16]hexathiacyclooctadecine; hexabenzo[b,e,h,k,n,q][1,4,7,10,13,16]hexathiacyclooctadecine; and 1,4,10,13-tetrathia-7,16-diazacyclooctadecane.

In some embodiments of the complexes disclosed herein, the metal cation has a charge of +1. In some embodiments, the metal cation is a cation of Ag or Pd. In some embodiments, the metal cation is a cation of Ag.

In some embodiments of the complexes disclosed herein, the metal cation has a charge of +2. In some embodiments, the metal cation is a cation of Ca, Cd, Co, Cr, Cu, Er, Fe, Hg, Mg, Mn, Nb, Ni, Pb, Pd, Sc, Sn, Sr, V, or Zn. In some embodiments, the metal cation is a cation of Mg, Fe, Hg, Sr, Sn, Ca, Cd, Zn, Co, Cu, Pb, Ni, Sc, V, Cr, or Mn. In some embodiments, the metal cation is a cation of Ni, Zn, Cu, Pb, or Co. In some embodiments, the metal cation is a cation of Ca, Cu, or Zn. In some embodiments, the metal cation is a cation of Cu. In some embodiments, the metal cation is a cation of Fe, Ni, Zn, Co, Sc, V, Cr, or Mn. In some embodiments, the metal cation is a cation of Ni, Zn, Co, Sc, V, Cr, or Mn. In some embodiments, the metal cation is a cation of Pd, Nb, Hg, and Er. In some embodiments, the metal cation is a polycation of Hg (i.e., Hg₂, Hg₃, or Hg₄).

In some embodiments of the complexes disclosed herein, the metal cation has a charge of +3. In some embodiments, the metal cation is a cation of Ce, Dy, Er, Eu, Fe, Gd, Ho, La, Lu, Nb, Nd, Pm, Pr, Sm, Tb, Tm, or Yb. In some embodiments, the metal cation is a cation of Fe, La, Ce, Pr, Nd, Pm, Sm, Eu, Gd, Tb, Dy, Ho, Er, Tm, Yb, or Lu. In some embodiments, the metal cation is a cation of Ac, Th, Pa, U, Np, Pu, Am, Cm, Bk, Cf, Es, Fm, Md, No, or Lr. In some embodiments, the metal cation is a cation of Nb or Er.

### Ionic Liquid Mixtures

In some embodiments disclosed herein, the ionic liquid mixture comprises an ionic liquid and a metal-chelating group. In some embodiments, the metal-chelating group is selected from the group consisting of an ethylaminediacetic acid moiety, a crown ether, a dithizone, a hydroxyquinoline, 2-thenoyltrifluoroacetone, a thiosalicylate, a salicylate, a thiocarbamate, a dithiocarbamate, an alkanolamine, a thioglycolate, an aza-crown ether, and a thia-crown ether.

In some embodiments, the IL mixture comprises an ethylaminediacetic acid moiety as a metal-chelating group. In some embodiments, the IL mixture comprises a metal-chelating group comprising

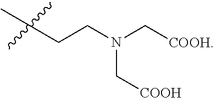 [SMILES: *CCN(CC(=O)O)CC(=O)O]

In some embodiments, the IL mixture comprises ethylenediaminetetra-acetic acid (EDTA), N-(2-hydroxyethyl)ethylenediaminetriacetic acid (HEDTA), or diethylenetriaminepentaacetic acid (DTPA).

In some embodiments, the IL mixture comprises a crown ether as a metal-chelating group. In some embodiments, the IL mixture comprises a crown ether selected from the group consisting of 12-crown-4; 2-hydroxymethyl-12-crown-4; 15-crown-5; 2-aminomethyl-15-crown-5; 2-hydroxymethyl-15-crown-5; 4′-aminobenzo-15-crown-5; 4′ -formylbenzo-15-crown-5; 4′-nitrobenzo-15-crown-5; 2,3-naphtho-15-crown-5; benzo-15-crown-5; 4′-carboxybenzo-15-crown-5; dibenzo-15-crown-5; 18-crown-6; 2-aminomethyl-18-crown-6; benzo-18-crown-6; 2-hydroxymethyl-18-crown-6; 4′-aminobenzo-18-crown-6; dicyclohexano-18-crown-6; dibenzo-18-crown-6; 4′-aminodibenzo-18-crown-6; 4′,4″(5″)-di-tert-butyldibenzo-18-crown-6; 4′,4″(5″)-di-tert-butyldicyclohexano-18-crown-6; (18-crown-6)-2,3,11,12-tetracarboxylic acid, dibenzo-21-crown-7; dibenzo-24-crown-8; and dibenzo-30-crown-10.

In some embodiments, the IL mixture comprises a dithizone as a metal-chelating group. In some embodiments, the IL mixture comprises

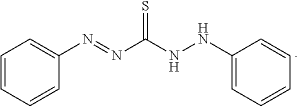 [SMILES: S=C(/N=N/c1ccccc1)NNc1ccccc1]

In some embodiments, the IL mixture comprises a hydroxyquinoline as a metal-chelating group. In some embodiments, the IL mixture comprises

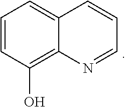 [SMILES: Oc1cccc2cccnc12]

In some embodiments, the IL mixture comprises 2-thenoyltrifluoroacetone as a metal-chelating group. In some embodiments, the IL mixture comprises

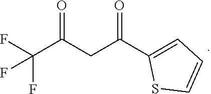 [SMILES: O=C(CC(=O)C(F)(F)F)c1cccs1]

In some embodiments, the IL mixture comprises a thiosalicylate or thiosalicylic acid as a metal-chelating group. In some embodiments, the IL mixture comprises

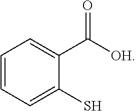

In some embodiments, the IL mixture comprises a salicylate or salicylic acid as a metal-chelating group. In some embodiments, the IL mixture comprises

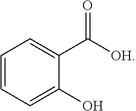

In some embodiments, the IL mixture comprises a thiocarbamate as a metal-chelating group. In some embodiments, the IL mixture comprises a dithiocarbamate. In some embodiments, the IL mixture comprises a dithiocarbamate selected from the group consisting of diethyldithiocarbamate, disulfiram, emetine dithiocarbamate, hexamethylene dithiocarbamic acid, 4-methylpiperidine dithiocarbamate, morpholine-4-carbodithioic acid, pentamethylene dithiocarbamate, phenylpiperazine dithiocarbamate, piperazine-dithiocarbamate, piperidine-1-carbodithioic acid, and pyrrolidine dithiocarbamate.

In some embodiments, the IL mixture comprises an alkanolamine as a metal-chelating group. In some embodiments, the IL mixture comprises an alkanolamine selected from the group consisting of monoethanolamine (MEA); 3-amino-1-propanol; diethanolamine (DEA); diisopropanolamine; 1-amino-2-propanol; 2-amino-2-methyl-1-propanol; 2-amino-2-ethyl-1,3-propanediol; 2-amino-2-hydroxymethyl-1,3-propanediol; 2-amino-1-butanol; 2-((2-aminoethyl)amino)ethan-1-ol; and 2,2′-(ethane-1,2-diylbis(oxy))bis(ethan-1-amine). In some embodiments, the IL mixture comprises an alkanolamine selected from the group consisting of

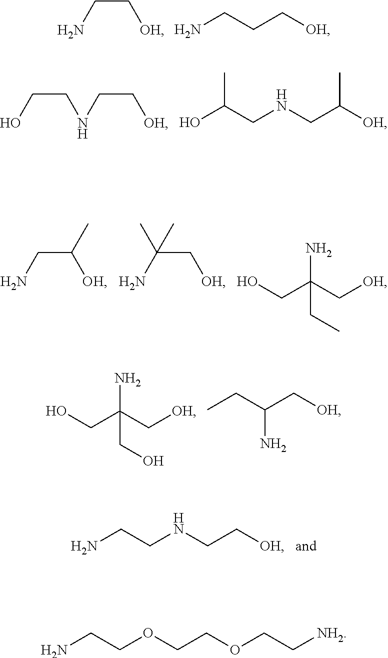

In some embodiments, the IL mixture comprises a thioglycolate as a metal-chelating group. In some embodiments, the IL mixture comprises

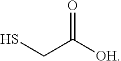

In some embodiments, the IL mixture comprises a thioglycolate selected from the group consisting of ammonium thioglycolate, sodium thioglycolate, methyl thioglycolate, and ethyl thioglycolate.

In some embodiments, the IL mixture comprises an aza-crown ether as a metal-chelating group. In some embodiments, the IL mixture comprises an aza-crown ether selected from the group consisting of 1-aza-12-crown-4; 1,7-diaza-12-crown-4; 4,10-diaza-12-crown-4; 1-aza-15-crown-5; 4,10-diaza-15-crown-5; N-phenylaza-15-crown-5; 1-aza-18-crown-6; 4,13-diaza-18-crown-6; 7,16-dibenzyl-1,4,10,13-tetraoxa-7,16-diazacycloocta-decane; 4,7,13,16,21-pentaoxa-1,10-diazabicyclo[8.8.5]tricosane; 4,7,13,16,21,24-hexaoxa-1,10-diazabicyclo[8.8.8]hexacosane; 5,6-benzo-4,7,13,16,21,24-hexaoxa-1,10-diazabicyclo[8.8.8]hexacos-5-ene; 5,6,14,15-dibenzo-1,4-dioxa-8,12-diazacyclopentadeca-5,14-diene; 1,4,10,13-tetraoxa-7,16-diazacyclooctadecane-1,4,7,10-tetraacetic acid; 1,4,7-triazacyclononane; 1,4,7-trimethyl-1,4,7-triazacyclononane; cyclen; hexacyclen; 1,4,7,10-tetraazacyclododecane; tri-tent-butyl 1,4,7,10-tetraazacyclododecane-1,4,7-triacetate; tri-tent-butyl 1,4,7,10-tetraazacyclododecane-1,4,7,10-tetraacetate; 1,4,8,12-tetraazacyclo-pentadecane; 1,4,8,11-tetraazacyclotetradecane; 1,4,8,11-tetraazacyclotetradecane-5,7-dione; 1,4,8,11-tetramethyl-1,4,8,11-tetraazacyclotetradecane; and 1,4,10,13-tetrathia-7,16-diazacyclooctadecane.

In some embodiments, the IL mixture comprises a thia-crown ether as a metal-chelating group. In some embodiments, the IL mixture comprises a thia-crown ether selected from the group consisting of 1,4,7-trithiacyclononane; 3,6,9-trithia-1(2,5)-thiophenacyclodecaphane; 1,4,7,10-tetrathiacyclododecane; 1,4,8,11-tetrathiacyclotetradecane; 1,4,7,10,13-pentathiacyclopentadecane; 1,5,9,13-tetrathiacyclohexadecane; 1,5,9,13-tetrathiacyclohexadecane-3,11-diol; 1,4,7,10,13,16-hexathiacyclooctadecane; 2,3,5,6,8,9,11,12,14,15-decahydrobenzo[b][1,4,7,10,13,16]hexathiacyclooctadecine; hexabenzo[b,e,h,k,n,q][1,4,7,10,13,16]hexathiacyclooctadecine; and 1,4,10,13-tetrathia-7,16-diazacyclooctadecane.

In some embodiments of the ionic liquid mixtures disclosed herein, the IL does not comprise an alkylethylenediaminium cation.

In some embodiments of the ionic liquid mixtures disclosed herein, the ionic liquid comprises a cation and an anion; and the cation is represented by the following structural formula I:

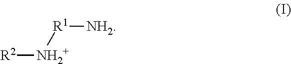

The variables in Formula I may be further selected as described above and below.

In some embodiments of the ionic liquid mixtures disclosed herein, the ionic liquid comprises a cation and an anion; and the cation is represented by the following structural formula II:

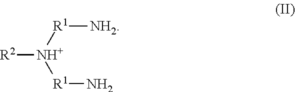

The variables in Formula II may be further selected as described above and below.

### Exemplary Embodiments of Variables in Structural Formulas I and II

In some embodiments of structural Formula I or II, n is 3. In some embodiments, n is 2. The remainder of the variables in structural Formula I or II may be selected as described above or below.

In some embodiments of structural Formula I or II, m is 1, 2, 3, or 4. In some embodiments, m is 5, 6, or 7. In some embodiments, m is 8, 9, or 10. In some embodiments, m is 1. In some embodiments, m is 4. In some embodiments, m is 6. The remainder of the variables in structural Formula I or II may be selected as described above or below.

In some embodiments of structural Formula I or II, R is F. In some embodiments, R is, for each instance independently, C₁-C₃ alkyl. In some embodiments, R is, for each instance independently, C₁-C₃ fluoroalkyl. In some embodiments, R is H. The remainder of the variables in structural Formula I or II may be selected as described above or below.

In some embodiments of structural Formula I or II, R′ is F. In some embodiments, R′ is C₁-C₈ alkyl. In some embodiments, R′ is C₁-C₈ fluoroalkyl. In some embodiments, R′ is H. The remainder of the variables in structural Formula I or II may be selected as described above or below.

In some embodiments of structural Formula I or II, R″ is F. In some embodiments, R″ is C₁-C₃ alkyl. In some embodiments, R″ is C₁-C₃ fluoroalkyl. In some embodiments, R″ is C₁-C₃ alkyloxy. In some embodiments, R″ is C₁-C₃ fluoroalkyloxy. In some embodiments, R″ is C₆-C₁₀ aryl. In some embodiments, R″ is C₂-C₈ alkenyl. In some embodiments, R″ is C₂ alkenyl. In some embodiments, R″ is C₂-C₈ fluoroalkenyl. In some embodiments, R″ is H.

In some embodiments, R″ is C₆-C₁₀ aryl, C₂-C₈ alkenyl or C₂-C₈ fluoroalkenyl; wherein each instance of C₆-C₁₀ aryl is substituted with one, two, three, four or five substituents independently selected from the group consisting of C₂-C₈ alkenyl or C₂-C₈ fluoroalkenyl.

In some embodiments, when R″ is C₆-C₁₀ aryl, it is unsubstituted.

In some embodiments, when R″ is C₆-C₁₀ aryl, it is substituted. In some embodiments, when R″ is C₆ aryl, it is substituted.

In some embodiments, the one or more substituents on R″ are independently selected from F, C₁-C₃ alkyl, and C₁-C₃ fluoroalkyl. In some embodiments, the one or more substituents on R″ are independently selected from C₁-C₃ alkyl. In some embodiments, the one or more substituents on R″ are independently selected from C₂-C₈ alkenyl or C₂-C₈ fluoroalkenyl. In some embodiments, the one or more substituents on R″ are independently selected from C₂-C₈ alkenyl. In some embodiments, the one or more substituents on R″ are independently C₂ alkenyl. In some such embodiments, R″ is substituted with one substituent selected from the group consisting of F, C₁-C₃ alkyl, C₁-C₃ fluoroalkyl, C₁-C₃ alkyloxy, and C₁-C₃ fluoroalkyloxy. In some such embodiments, R″ is substituted with two substituents selected from the group consisting of F, C₁-C₃ alkyl, C₁-C₃ fluoroalkyl, C₁-C₃ alkyloxy, and C₁-C₃ fluoroalkyloxy. In some such embodiments, R″ is substituted with three such substituents. In some such embodiments, R″ is substituted with four such substituents. In some such embodiments, R″ is substituted with five such substituents. The remainder of the variables in structural Formula I or II may be selected as described above or below.

In some embodiments of structural Formula I or II, n is 2; and R is H. In some embodiments, m is 1; R″ is substituted C₆ aryl, wherein the substituent on R″ is C₂ alkenyl. The remainder of the variables in structural Formula I or II may be selected as described above or below.

In some embodiments of structural Formula I or II, m is 4; and R″ is H. In some embodiments, R² is butyl. In some embodiments, m is 6; and R″ is H. In some embodiments, R² is 2-ethylhexyl. In some embodiments, R² is hexyl. The remainder of the variables in structural Formula I or II may be selected as described above or below.

### Articles

In some embodiments of the ILs, IL complexes, and mixtures comprising an IL and a metal-chelating group disclosed herein, the IL, IL complex, or mixture comprising an IL and a metal-chelating group is incorporated into an article. In some embodiments, for example, the article is selected from filters (e.g., hand-held water filters), membranes, packing materials (e.g., for foods, agriculture, paints, etc.), flow cells, filter gaskets, gloves, masks, garments, wound dressings, implants, catheters, and other medical devices. In some embodiments, the article is sterile.

### Nanoparticles

Also provided herein are nanoparticles. In some embodiments, the nanoparticles are functionalized with the ionic liquids, ionic liquid complexes, and ionic liquid mixtures disclosed herein. In some embodiments, an ionic liquid coating comprising ionic liquids, ionic liquid complexes, and ionic liquid mixtures surrounds magnetic nanoparticles. In some embodiments, the nanoparticles may be concentrated using a magnetic field (employing magnetic nanoparticles). In some embodiments, the plurality of nanoparticles comprise an ionic liquid complex as disclosed herein. In some embodiments, the plurality of nanoparticles comprise an ionic liquid mixture as disclosed herein.

In some embodiments, extraction occurs using electrochemistry.

### Definitions

Unless otherwise defined herein, scientific and technical terms used in this application shall have the meanings that are commonly understood by those of ordinary skill in the art. Generally, nomenclature used in connection with, and techniques of, chemistry described herein, are those well-known and commonly used in the art.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkanolamine” refers to a moeity comprising an amino group, a hydroxy group, and an alkyl group.

The term “alkoxy” refers to an alkyl group, having an oxygen attached thereto. Representative alkoxy groups include methoxy, trifluoromethoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Typically, a straight chained or branched alkenyl group has from 1 to about 20 carbon atoms, preferably from 1 to about 10 unless otherwise defined. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

An “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. In some embodiments, the alkyl group has from 1 to 8 carbon atoms, from 1 to 6 carbon atoms, from 1 to 4 carbon atoms, or from 1 to 3 carbon atoms. Examples of straight chained and branched alkyl groups include methyl, ethyl, n-propyl, iso-propyl, n-butyl, sec-butyl, tert-butyl, pentyl, hexyl, pentyl and octyl.

Moreover, the term “alkyl” as used throughout the specification, examples, and claims is intended to include both “unsubstituted alkyls” and “substituted alkyls”, the latter of which refers to alkyl moieties having substituents replacing a hydrogen on one or more substitutable carbons of the hydrocarbon backbone. Such substituents, if not otherwise specified, can include, for example, a halogen (e.g., fluoro), a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C₁₋₆ alkyl, C₃₋₆ cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y} alkyl” refers to substituted or unsubstituted saturated hydrocarbon groups, including straight-chain alkyl and branched-chain alkyl groups that contain from x to y carbons in the chain, including haloalkyl groups. Preferred haloalkyl groups include trifluoromethyl, difluoromethyl, 2,2,2-trifluoroethyl, and pentafluoroethyl. C₀ alkyl indicates a hydrogen where the group is in a terminal position, a bond if internal. The terms “C_{2-y} alkenyl” and “C_{2-y} alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “arylthio”, as used herein, refers to a thiol group substituted with an alkyl group and may be represented by the general formula arylS—.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Typically, a straight chained or branched alkynyl group has from 1 to about 20 carbon atoms, preferably from 1 to about 10 unless otherwise defined. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

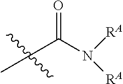

wherein each R^{A} independently represent a hydrogen or hydrocarbyl group, or two R^{A} are taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

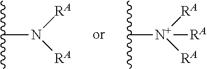

wherein each R^{A} independently represents a hydrogen or a hydrocarbyl group, or two R^{A} are taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 6- or 20-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “aza-crown ether” is used herein to refer to a ring system comprising at least one nitrogen atom and several ether groups. In some embodiments, the aza-crown ether refers to a ring system comprising nitrogen atoms and carbon atoms.

The term “carbamate” is art-recognized and refers to a group

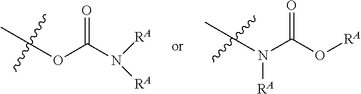

wherein each R^{A} independently represent hydrogen or a hydrocarbyl group, such as an alkyl group, or both R^{A} taken together with the intervening atom(s) complete a heterocycle having from 4 to 8 atoms in the ring structure.

The terms “carbocycle”, and “carbocyclic”, as used herein, refers to a saturated or unsaturated ring in which each atom of the ring is carbon. Preferably, a carbocylic group has from 3 to 20 carbon atoms. The term carbocycle includes both aromatic carbocycles and non-aromatic carbocycles. Non-aromatic carbocycles include both cycloalkane rings, in which all carbon atoms are saturated, and cycloalkene rings, which contain at least one double bond. “Carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be susbstituted at any one or more positions capable of bearing a hydrogen atom.

The term “crown ether”, as used herein, refers to a ring system containing several ether groups.

A “cycloalkyl” group is a cyclic hydrocarbon which is completely saturated. “Cycloalkyl” includes monocyclic and bicyclic rings. Preferably, a cycloalkyl group has from 3 to 20 carbon atoms. Typically, a monocyclic cycloalkyl group has from 3 to about 10 carbon atoms, more typically 3 to 8 carbon atoms unless otherwise defined. The second ring of a bicyclic cycloalkyl may be selected from saturated, unsaturated and aromatic rings. Cycloalkyl includes bicyclic molecules in which one, two or three or more atoms are shared between the two rings. The term “fused cycloalkyl” refers to a bicyclic cycloalkyl in which each of the rings shares two adjacent atoms with the other ring. The second ring of a fused bicyclic cycloalkyl may be selected from saturated, unsaturated and aromatic rings. A “cycloalkenyl” group is a cyclic hydrocarbon containing one or more double bonds.

The term “carbocyclylalkyl”, as used herein, refers to an alkyl group substituted with a carbocycle group.

The term “carbonate”, as used herein, refers to a group —OCO₂-R^{A}, wherein R^{A} represents a hydrocarbyl group.

The term “carboxy”, as used herein, refers to a group represented by the formula —CO₂H.

The term “ester”, as used herein, refers to a group —C(O)OR^{A} wherein R^{A} represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The term “heteroalkyl”, as used herein, refers to a saturated or unsaturated chain of carbon atoms and at least one heteroatom, wherein no two heteroatoms are adjacent.

The terms “heteroaryl” and “hetaryl” include substituted or unsubstituted aromatic single ring structures, preferably 5- to 20-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 20-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “heterocyclylalkyl”, as used herein, refers to an alkyl group substituted with a heterocycle group.

The term “hydrocarbyl”, as used herein, refers to a group that is bonded through a carbon atom, wherein that carbon atom does not have a ═O or ═S substituent. Hydrocarbyls may optionally include heteroatoms. Hydrocarbyl groups include, but are not limited to, alkyl, alkenyl, alkynyl, alkoxyalkyl, aminoalkyl, aralkyl, aryl, aralkyl, carbocyclyl, cycloalkyl, carbocyclylalkyl, heteroaralkyl, heteroaryl groups bonded through a carbon atom, heterocyclyl groups bonded through a carbon atom, heterocyclylakyl, or hydroxyalkyl. Thus, groups like methyl, ethoxyethyl, 2-pyridyl, and trifluoromethyl are hydrocarbyl groups, but substituents such as acetyl (which has a ═O substituent on the linking carbon) and ethoxy (which is linked through oxygen, not carbon) are not.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are six or fewer non-hydrogen atoms in the substituent. A “lower alkyl”, for example, refers to an alkyl group that contains six or fewer carbon atoms. In some embodiments, the alkyl group has from 1 to 6 carbon atoms, from 1 to 4 carbon atoms, or from 1 to 3 carbon atoms. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

In the phrase “poly(meta-phenylene oxides)”, the term “phenylene” refers inclusively to 6-membered aryl or 6-membered heteroaryl moieties. Exemplary poly(meta-phenylene oxides) are described in the first through twentieth aspects of the present disclosure.

The term “silyl” refers to a silicon moiety with three hydrocarbyl moieties attached thereto.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. Moieties that may be substituted can include any appropriate substituents described herein, for example, acyl, acylamino, acyloxy, alkoxy, alkoxyalkyl, alkenyl, alkyl, alkylamino, alkylthio, arylthio, alkynyl, amide, amino, aminoalkyl, aralkyl, carbamate, carbocyclyl, cycloalkyl, carbocyclylalkyl, carbonate, ester, ether, heteroaralkyl, heterocyclyl, heterocyclylalkyl, hydrocarbyl, silyl, sulfone, or thioether. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C₁₋₆ alkyl, C₃₋₆ cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

The term “sulfonate” is art-recognized and refers to the group SO₃H, or a pharmaceutically acceptable salt thereof.

The term “sulfone” is art-recognized and refers to the group —S(O)₂-R^{A}, wherein R^{A} represents a hydrocarbyl.

The term “thia-crown ether” is used herein to refer to a ring system comprising at least one sulfur atom and several ether groups. In some embodiments, the thia-crown ether refers to a ring system comprising sulfur atoms and carbon atoms.

The term “thioether”, as used herein, is equivalent to an ether, wherein the oxygen is replaced with a sulfur.

### EXAMPLES

The invention now being generally described, it will be more readily understood by reference to the following examples, which are included merely for purposes of illustration of certain aspects and embodiments of the present invention, and are not intended to limit the invention.

### Example 1: Synthesis and Physical Characterization of an Ionic Liquid Comprising a Metal-Chelating Group

### Exemplary Aza-Crown Ether IL

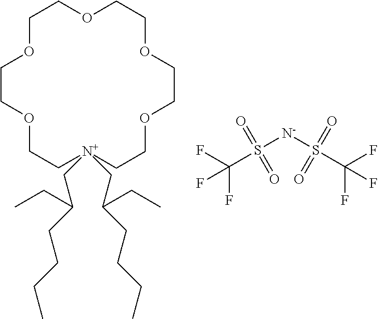 [SMILES: CCCCC(CC)C[N+]1(CC(CC)CCCC)CCOCCOCCOCCOCCOCC1 ; O=S(=O)([N-]S(=O)(=O)C(F)(F)F)C(F)(F)F]

16,16-di(octan-3-yl)-1,4,7,10,13-pentaoxa-16-azacyclooctadecan-16-ium was synthesized according to the following procedure.

Route A: 3-(chloromethyl)heptane was added to a solution of 1-aza-18-crown-6 as a solvent. The mixture was heated to reflux and was reacted for about 24 hours. 16,16-di(octan-3-yl)-1,4,7,10,13 -pentaoxa-16-azacyclooctadecan-16-ium chloride salt was obtained.

Route B: 3-(chloromethyl)heptane (10-30 equivalents) was added to a solution of 1-aza-18-crown-6 (1 equivalent). The mixture was heated to reflux and was reacted for about 24 hours. 16,16-di(octan-3-yl)-1,4,7,10,13-pentaoxa-16-azacyclooctadecan-16-ium chloride salt was obtained.

1,1,1-trifluoro-N-((trifluoromethyl)sulfonyl)methanesulfonamide (bis(trifluoromethane-sulfonyl)imide, TFSIH) was reacted with silver(I) oxide in a separate flask to form silver bis(trifluoro-methanesulfonyl)imide (AgTFSI). AgTFSI was dissolved in acetonitrile. The aza-crown halide salt was added to the AgTFSI solution to precipitate silver halide. Thus, an exemplary aza-crown TFSI ionic liquid was formed.

### Exemplary Ethylenediamine IL

### 2-ethylhexyl(ethylenediaminium) bis(trifluoroethanesulfonyl)amide ([eth-hex-en][Tf₂N]) synthesis

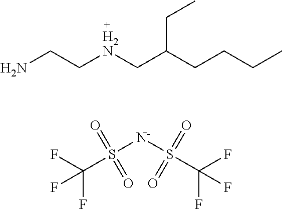

To 5.0 moles of neat ethylenediamine (300.5 g), 1 mole of 2-ethylhexyl bromide (193.5 g) was added dropwise for three hours with vigorous stirring. The reaction mixture was then slowly heated to 50° C. and was allowed to stir under reflux for 12 h. After the reaction, unreacted ethylenediamine was removed under vacuum. The liquid solution was washed three times with 2 M sodium hydroxide, and the organic phase was collected and dried over anhydrous magnesium sulfate. 2-ethylhexylethylenediamine was purified by vacuum distillation (20 mtorr) in the temperature range of 90-150° C. for about 6 hours. The fraction collected during this time was a single substituted ethylenediamine, the yield was 80%. Next, a solution of 0.8 mole of bis(trifluoromethane)sulfonimide (224.8 g) in 300 mL of THF was added dropwise over a period of 1 h to a solution of 0.8 moles of the 2-ethylhexylethylenediamine (138.65 g) in 300 ml of THF (exothermic reaction). After 2 h of reaction, THF was removed under vacuum, and the yield was 97%.

### 2-ethylhexyl(tetramethylethylenediaminium) bis(trifluoroethanesulfonyl)amide ([eth-hex-tmeda][Tf₂N]) synthesis

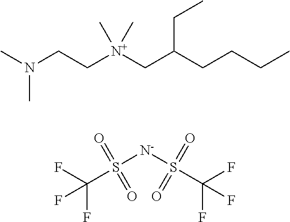 [SMILES: CCCCC(CC)C[N+](C)(C)CCN(C)C ; O=S(=O)([N-]S(=O)(=O)C(F)(F)F)C(F)(F)F]

Route A: 3-(chloromethyl)heptane (1 equivalent) was added to a solution of tetramethylethylenediamine (3-5 equivalents). The mixture was heated to reflux and was reacted for about 24 hours. The produce was then purified by removing excess alkyl halide under reduced pressure. N-(2-(dimethylamino)ethyl)-2-ethyl-N,N-dimethylhexan-1-aminium chloride salt was obtained.

TFSIH was reacted with silver(I) oxide in a separate flask to form silver bis(trifluoro-methanesulfonyl)imide (AgTFSI). AgTFSI was dissolved in acetonitrile. The exemplary ethylenediamine halide salt was added to the AgTFSI solution to precipitate silver halide. Thus, an exemplary ethylenediamine TFSI ionic liquid was formed.

Route B: A mixture of 0.5 mol N,N,N′,N′-tetramethylethylenediamine (58.1 g) and 0.1 mol of 2-ethylhexyl bromide (19.3 g) was refluxed for 12 h. Unreacted tetramethylethylenediamine was removed under vacuum, and the yield of 2-ethylhexyltetramethylethylenediamine was ˜100%. Next, a solution of 0.1 mol of silver bis(trifluoromethane)sulfonamide was made by vigorously stirring a suspension of 0.05 mol silver (I) oxide (11.57 g) and 0.1 mol bis(trifluoromethane)sulfonamide (28.1 g) in isopropanol. After 0.5 h of reaction, the solution of silver bis(trifluoromethane)sulfonamide was added dropwise to a solution of 0.1 mol of 2-ethylhexyltetramethylethylenediamine (30.9 g) in isopropanol with continuous stirring. The resulting silver bromide was removed by a sequence of three centrifugations of the reaction mixture and decantation. Isopropanol was then removed by vacuum.

The protons on the ammonium moiety in 2-ethylhexyl(ethylenediaminium) bis(trifluoroethanesulfonyl)amide are readily reduced to hydrogen molecules in reducing environments, such as the cathode half-cell in the electroplating cell. This limited its electrochemical regeneration to weakly reducing metals such as silver, copper and lead. Therefore, to broaden the range of metals that could be electroplated, a structurally similar IL was synthesized, [eth-hex-tmeda][Tf₂N], which replaces acidic protons on the ethylenediamine moiety with methyl groups. These survive higher reducing potentials than the protons on [eth-hex-en][Tf₂N], and hence allow the electroplating of more reducing metals, which regenerates the IL without its degradation.

### Exemplary Thioglycolate IL³²

### Butylsulfanyl Acetic Acid (C₄SAcH) Precursor

To a solution of thioglycolic acid (15.0 g, 0.16 mol) in ethanol (50 mL), potassium hydroxide (27.4 g, 0.49 mol) in water (20 mL) was added. After complete dissolution, 1-iodobutane (32.7 g, 0.18 mol) was added. The solution was stirred at reflux for 3 h. After cooling to room temperature, ethanol was distilled off and the remaining aqueous solution was acidified with conc. HCl to pH 1 and extracted with diethyl ether (4×30 mL). The combined organic layers were washed with water (2×20 mL), dried over MgSO₄, filtered off and concentrated to dryness. The obtained product was dried in vacuo yielding an orange oil. Yield: 21.6 g (90%). ¹H NMR (400.20 MHz, CDCl₃): δ 3.26 (s, 2H; SCH₂COOH), 2.67 (t, J=7 Hz, 2H; SCH₂CH₂), 1.55-1.65 (m, 2H; —CH₂—), 1.37-1.48 (m, 2H; —CH₂CH₃), 0.93 ppm (t, J=7 Hz, 3H; −CH₃). ¹³C NMR (100.63 MHz, CDCl3): δ 175.3, 33.5, 32.5, 31.0, 21.8, 13.6 ppm.

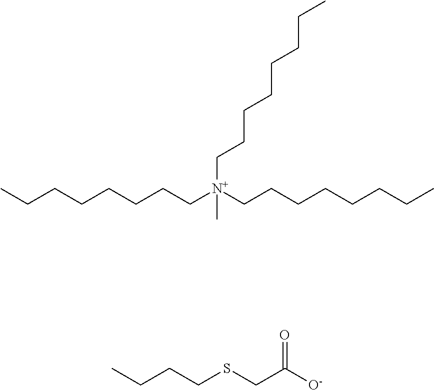

### Methyltrioctylammonium butylsulfanyl acetate [N₁₈₈₈][C₄SAc]

Butylsulfanyl acetic acid (1.5 g, 0.01 mol) in methanol (30 mL) and [N₁₈₈₈][MC] (4.4 g, 0.01 mol) in methanol (20 mL) were stirred for 3 h at room temperature. The solvent was distilled off and the product was dried in vacuo at 40° C. for 2 days. Yield: 5.1 g (100%). ¹H NMR (400.20 MHz, CDCl₃):δ 3.32-3.43 (m, 6H, N(CH₂)₃—), 3.27 (s, 3H; NCH₃), 3.22 (s, 2H; SCH₂COO⁻), 2.52-2.60 (t, J=8 Hz, 2H; SCH₂CH₂), 1.49-1.69 (m, 8H; —CH₂—), 1.16-1.42 (m, 32H; —CH₂—CH₂—), 0.79-0.91 ppm (m, 12H; —CH₃). ¹³C NMR (100.63 MHz, CDCl3): δ 174.1, 61.2, 48.7, 39.2, 323.4, 31.8, 31.7, 29.2, 29.1, 26.4, 22.6, 22.45, 14.1, 13.9 ppm. IR (ATR, selected bands, vₘₐₓ): 2926, 2858, 1560, 1463, 1554 cm⁻¹. UV/Vis in MeOH, λ, nm (c, M⁻¹ cm⁻¹): 208 (1890). ESI-MS (pos) m/z: 368.3 [N₁₈₈₈]⁺. ESI-MS (neg) m/z: 147.0 [C₄SAc]⁻.

### Example 2: Metal Extraction by an Ionic Liquid Comprising a Metal-Chelating Group from Aqueous Solution and Regeneration of the Ionic Liquid

The extraction procedure was quite simple, and it was based on vigorous shaking of a fixed volume of the chosen metal aqueous solution with the ionic liquid by using a vortex shaker. The whole process was conducted in 15 mL plastic Falcon tubes to enable a quick and efficient phase separation after the process by centrifugation of the samples for 1 minute at 4000 rpm. After this, the phase separation was very clear, and it was easy to collect the aqueous phase for further ICP analysis.

Metal concentration analysis was conducted by using an Optima 8300 Inductively Coupled Plasma Optical Emission Spectrometer (ICP OES) from Perkin Elmer (USA). Analyses were conducted both in axial and radial mode, depending on the metal concentration, type of the measured metal and sample matrix effects. However, for most of the analyzed metals Limit of Detection (LOD) was equal about 0.25 mg/L.

### Breakthrough Profiles for [eth-hex-en][Tf₂N] Ionic Liquid

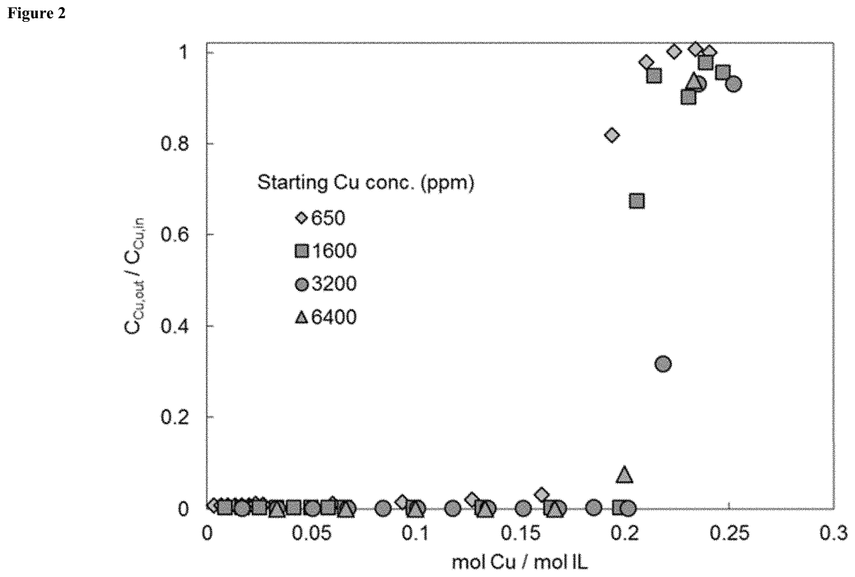

Extraction of various metal nitrates (Ag, Al, Ca, Co, Cu, Dy, Mg, Ni, Pb) at different concentrations from aqueous solution into the [eth-hex-en][Tf₂N] ionic liquid phase was investigated. This was done by contacting multiple aliquots of aqueous solutions of the metal ions with the IL until saturation of IL was achieved. In all cases, a complete removal of the metal ions was achieved. The concertation of metal ions in the aliquots after extraction was beyond the detection limits of the ICP-OES even up to 70% of IL saturation. After which the concentration of the solution started increasing gradually due to transport limitations posed by the increasing viscosity of the metal-saturated IL. This was proven by analyzing breakthrough profiles for the selected metals. First breakthrough profiles of copper at different initial concentrations were analyzed where fewer aliquots were needed for the concentrated solutions, shown in FIG. 2.

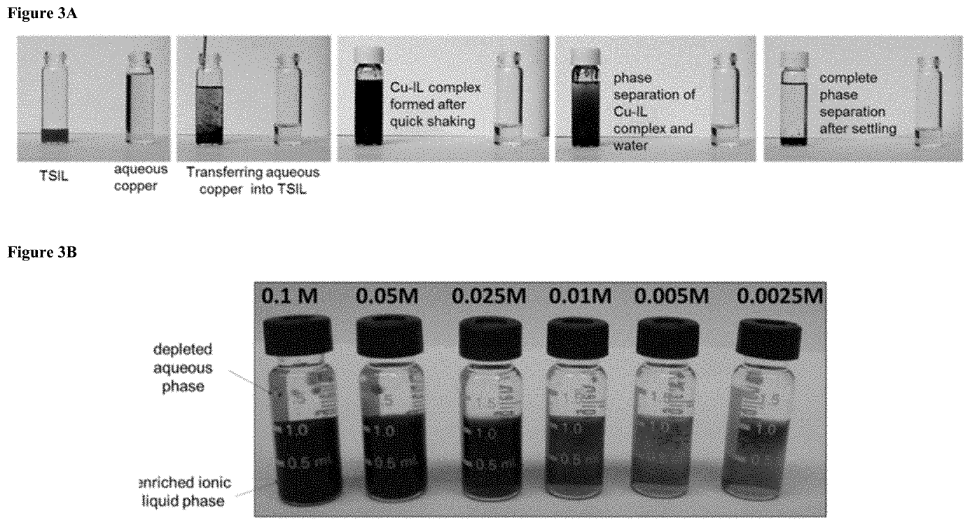

The complete removal of metal ions by [eth-hex-en][Tf₂N], especially at concentrations <70% saturation indicate that there is a very strong phase separation between the IL or metal-IL and the aqueous phase, i.e. high Nernst distribution coefficient in favor of the IL. FIG. 3A shows the steps of extraction of copper from 0.1 M Cu(NO₃)₂ solution. FIG. 3B shows the depleted aqueous phases and the enriched IL phases at different starting aqueous concentrations.

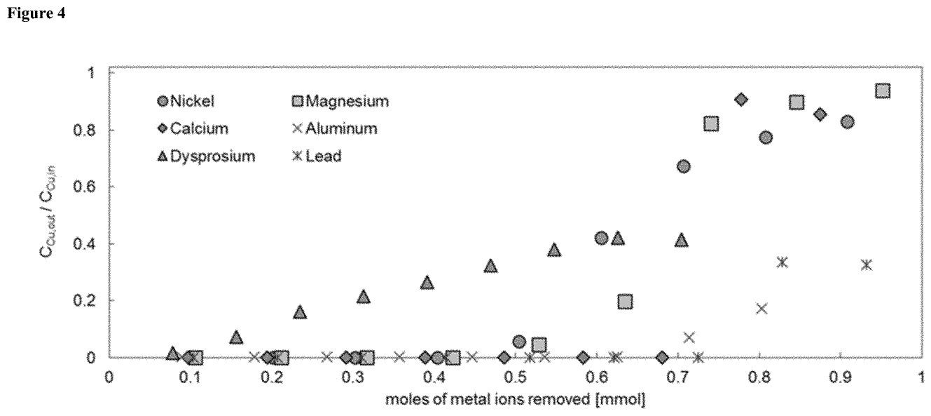

FIG. 4 shows the breakthrough profiles of six other metals with [eth-hex-en][Tf₂N]. Results presented in FIG. 4 shows that most metals exhibit a sharp breakthrough profile with complete removal of the metal via IL saturation. The only exception being the dysprosium profile, which shows a linear growth of dysprosium concentration in the aqueous phase after every cycle of extraction. Thus it can be assumed, that for this metal Nernst distribution coefficient has to be much less favorable than in the case of other metals. However, an important observation during this analysis was that in all of the cases, complexes of selected metals and [eth-hex-en][Tf₂N] remained in the IL phase, and neither crystallization nor solidification processes occurred.

Another type of the ionic liquid used during the presented studies [eth-hex-tmeda][Tf₂N], was slightly more problematic, due to a solidification process that occurs when the saturation of ionic liquids was higher than about 20%. Thus, there were no breakthrough profiles prepared for this ionic liquid; however, the efficiency of metals removal in many cases was relatively high and similar to [eth-hex-en][Tf₂N] results. Moreover, an important observation was that below 20% saturation [eth-hex-tmeda][Tf₂N] complex remained in the liquid phase, so it could be used in a similar way as [eth-hex-en][Tf₂N] but the regeneration process should be done at an earlier stage to avoid phase transition of the IL-metal complexes.

### Example 3: Electrochemical Measurements and Deposition

Electrochemical measurements were carried out using a VersaSTAT 3 potentiostat with VersaStudio software from Princeton Applied Research (USA). Cyclic voltammetry was conducted in a standard three-electrode glass cell with glassy carbon as the working electrode, 1 cm² platinum plate electrodes as the counter electrode and a Ag|AgNO₃reference electrode. The ionic liquid electrolyte was purged with nitrogen with gentle stirring for 30 min, and a nitrogen atmosphere was maintained during the electrochemical experiments. The temperature of the cell was controlled by immersing it into an oil bath.

### Chemical Regeneration of Ionic Liquids, Loaded with Selected Metals

When the ionic liquid was saturated by metal ions, it was regenerated by using two different methods. The first method was the commonly used chemical regeneration technique via diluted acid solutions. Here, 10% solutions of nitric acid or 5% solutions of hydrochloric acid were used. Ionic liquid after the extraction was shaken with the acid solution in a similar manner. After a few cycles, when the ionic liquid was completely regenerated, and there were no more metals migrating from ionic liquid to aqueous acid phase, ILs were washed three times by deionized water to wash out rest of the acid. Then the ionic liquid was ready for another extraction process.

To analyze the possibility of chemical regeneration, silver, cobalt, copper, and nickel have been studied, based on the previous analysis and the fact that they can be important metals in a number of industrial applications. The initial concentration of each metal in the aqueous phase was about 0.01 M and the volume was 20 mL, while the ionic liquid mass in each sample was 4 g, so after each cycle, assuming 100% regeneration, about 90% IL saturation should be achieved, based on theoretical calcuations. Actual initial concentrations of the selected metals were equal at about 0.01 M: 1164.8, 572.5, 663.9 and 547.3 ppm respectively for silver, cobalt, copper, and nickel. Moreover, chemical regeneration analyses were conducted by using two different types of acid as regenerating agents. First was 10% nitric acid solution that was added in 10 mL volume, to saturated ionic liquids after the extraction process and previous removal of the purified aqueous phase. Results of the chemical regeneration using 10% nitric acid are shown in Table 1.

TABLE 1
Chemical regeneration of both types of ILs using 10% HNO₃ solution.

| | | Metals concentration | | | | |
|---|---|---|---|---|---|---|
| | | after each extraction cycle [ppm] | | | | |
| | | 1^{st} | 2^{nd} | 3^{rd} | 4^{th} | 5^{th} |
| | metal | cycle | cycle | cycle | cycle | cycle |
| [eth-hex-en] | Ag | 2.36 | 2.79 | 3.34 | 3.50 | 18.66 |
| [Tf₂N] | Co | 67.13 | 186.66 | 187.01 | 101.86 | 293.21 |
| | Cu | −0.02 | −0.04 | 1.07 | 19.28 | 273.71 |
| | Ni | 125.82 | 139.58 | 3.79 | 47.40 | 262.79 |
| [eth-hex-tmeda] | Ag | 894.9 | 1062.7 | 1032.2 | 707.1 | 1104.8 |
| [Tf₂N] | Co | 574.2 | 561.6 | 549.1 | 546.1 | 592.1 |
| | Cu | 255.5 | 463.9 | 434.3 | 187.4 | 624.8 |
| | Ni | 618.6 | 604.1 | 448.5 | 366.9 | 605.9 |

Results in Table 1 show that it is possible to recycle both of the synthesized ILs by using nitric acid as a regenerating agent with different metals behaving differently. Another popular acid used in industrial metal stripping is hydrochloric acid, so another set of analyses was made by using 5% HCl as a regenerating agent, when all of the others conditions was exactly the same as in the case of HNO₃ regeneration. Table 2 shows the results of with HCl regeneration.

TABLE 2
Chemical regeneration of both types of ILs using 5% HCl solution.

| | | Metals concentration after | | |
|---|---|---|---|---|
| | | each extraction cycle [ppm] | | |
| | metal | 1^{st} cycle | 2^{nd} cycle | 3^{rd} cycle |
| [eth-hex-en][Tf₂N] | Ag | 2.59 | 1.98 | 2.57 |
| | Co | 157.65 | 277.8 | 415.3 |
| | Cu | −0.16 | 319.5 | 428.1 |
| | Ni | 235.2 | 248.7 | 533.6 |
| [eth-hex-tmeda][Tf₂N] | Ag | 979.3 | 982.4 | 1009.9 |
| | Co | 586.1 | 570.1 | 550.2 |
| | Cu | 308.7 | 356.3 | 356.2 |
| | Ni | 612.5 | 614.9 | 264.7 |

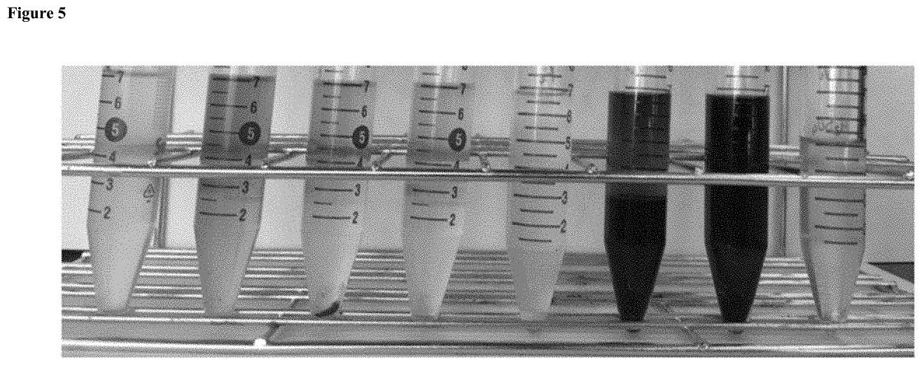

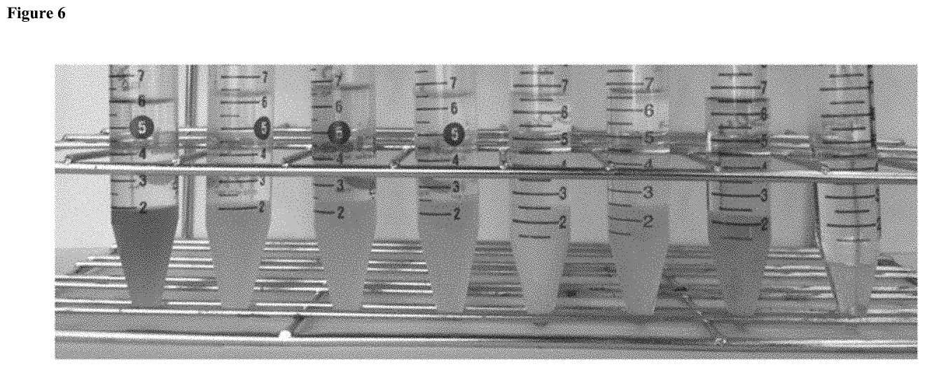

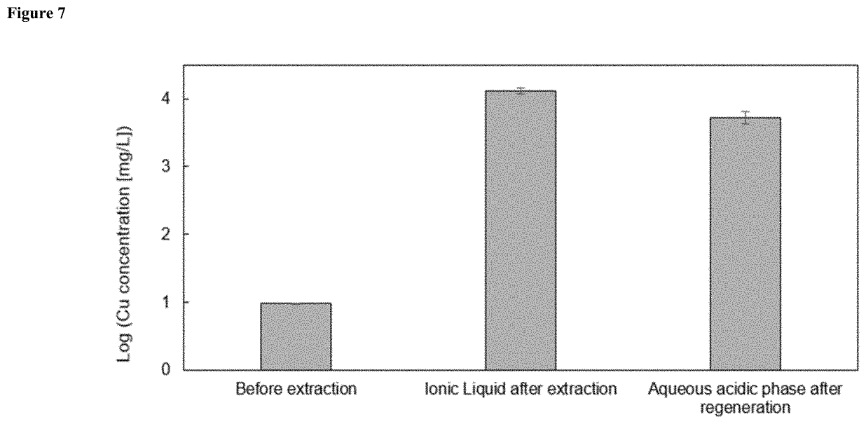

In the case of HCl regeneration, there were only three cycles conducted during the study, because after every cycle of extraction and regeneration, there were some losses in the amount of the ionic liquids. Thus, after three cycles less than 30% of the initial weight has left, so it was assumed that the experiment has to be aborted. Using hydrochloric acid is not an optimal way of chemical regeneration of the selected ionic liquids. Moreover, some more studies were conducted, verifying the results for a different HCl concentrations and trying to use some other acids like for example sulfuric acid, but in all of these cases there were more losses observed or there was no regeneration of ionic liquid at all. That is why it was concluded, that the preferred way to chemically regenerate both of the analyzed ILs is to use nitric acid as a regenerating agent. FIGS. 5 and 6 show the samples after acid regeneration of the ILs showing clearly that metals extracted in the previous process have been transferred to an aqueous acid phase (upper phase in all of the vials)

As observed in FIGS. 5 and 6, [eth-hex-en][Tf₂N] has extracted much more metals, thus the release to the water phase during the ILs regeneration step was also much clearer than in the case of [eth-hex-tmeda][Tf₂N]. Moreover, when 5% HCl was used to regenerate ILs, the aqueous solution above the ILs seems to be much darker and more intense, which can confirm that some part of ionic liquids is dissolved by HCl and migrating to the water phase. On the other hand, that effect in the case of [eth-hex-tmeda][Tf₂N] regeneration seems to be much less visible. However, it can be also related to a lower removal of the metals during the extraction step by this type of IL.

Another important aspect related with a chemical regeneration of the ILs was to concentrate metal ions by using ILs as a chelating agent and then, stripped it by a mentioned acid wash technique with a much smaller volume of the aqueous phase. Thus, it was possible to strongly increase the concentration of selected metals. In one of the experiment, 1 g of the [eth-hex-en][Tf₂N] IL was added to 1000 ml of 9.62 mg/L solution of copper nitrate. After 30 minutes of vigorous mixing, metal concentration in both phases were analyzed. In the aqueous phase, a copper level was below the detection limit of ICP-OES even in the axial configuration. On the other hand, the concentration of copper in the ionic liquid was about 12 944 mg/L. Next, a 2 ml of 10% nitric acid was added to a dried [eth-hex-en][Tf₂N], then mixed and centrifugated to obtain a phase separation. After that, copper concentration in aqueous acidic phase was also analyzed, and it was equal 5216 mg/L.

Using of described ionic liquid it is possible to increase metal concentration in the selected phase more than 500 times, which can be considered as one of the very important applications of the mentioned ILs in the micropollutants removal during the water and wastewater treatment processes. An important part is that, by using an HNO₃ regeneration techniques it is possible to concentrate selected metals not only by transferring them from aqueous to organic phase but after regeneration of organic phase with acid solution with a much smaller volume, it is possible to obtain about 500 higher concentration of metal in the water phase, after a simple 2 step process of extraction and regeneration.

### Direct Electrochemical Regeneration of Ionic Liquids

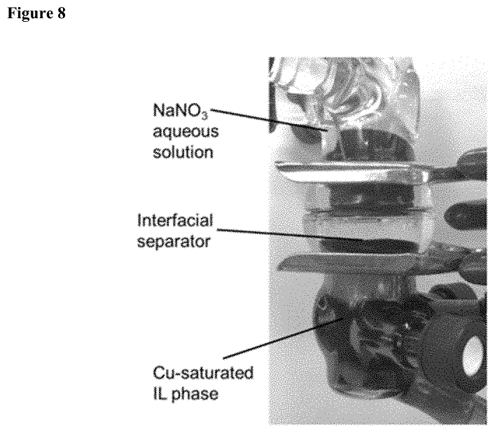

Electrochemical regeneration of the ILs via the electroplating of the metal ions presents a more efficient alternative to chemical regeneration. The second method of saturated ILs regeneration was direct electroplating of the chelated metals. In that method, the saturated ionic liquid was transferred to a 10 ml glass vial and different types of electrodes were used to plate out the metals from the ILs. Plating process was conducted in a vertically separated two-phase systems, where cathode was immersed in the lower organic phase and anode was suspended in the upper aqueous phase of 1 M sodium nitrate solution. On the anodic side of the system, oxygen evolution reaction (OER) took place; meanwhile, on the cathode, metal ions were reduced and plated on the cathode surface. The anode was Pd/C coated carbon felt electrode to allow for high-rate OER. The anodic aqueous solution was changed constantly to minimize pH change and re-extraction of metal ions. The interface between the anode aqueous electrolyte and the cathode IL (organic) electrolyte served as the separator (FIG. 8).

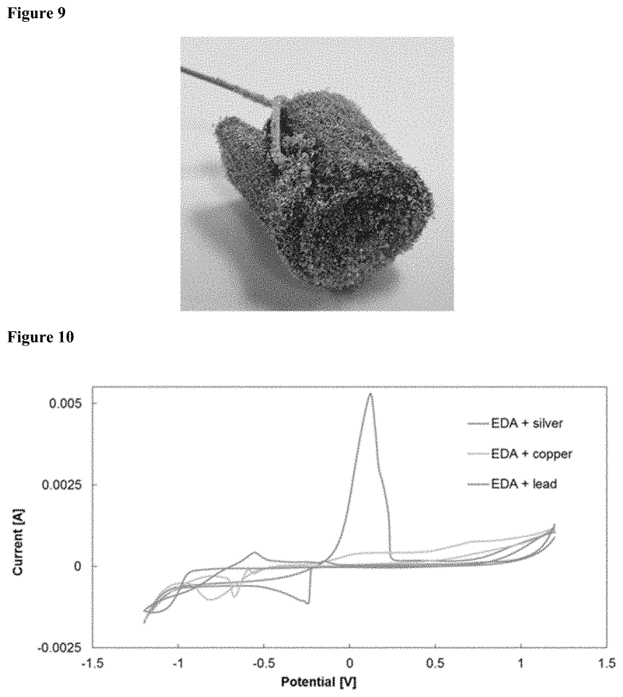

One unique aspect of these studies is that, after removal of the aqueous phase and drying of the IL the chelated metals can be deposited electrochemically in order to recycle the IL. Some advantages of using ionic liquids over aqueous or organic solvents are that there can be no evaporation (ILs have no vapor pressure), also it averts the need for supporting electrolytic salts for the electrochemical recovery step have been reported. Electrochemically-mediated ionic liquids that bind to both cupric ions and CO₂ can be employed in novel methods to scrub CO₂ from flue gas [27, 28]. Very few examples exist of electrodeposition from an IL with a metal-containing cation [29]. This is beneficial to metal deposition because the electroactive species can easily access the electrode surface as compared to the more common anionic metal complexes, which must travel against the electric field and compete with other cations under reductive conditions, thus improving energy efficiency of the process [27]. FIG. 9 shows a carbon electrode used during the electro regeneration of the [eth-hex-en][Tf₂N] IL after the two regeneration cycles of silver extraction.

The silver covering the whole surface of the electrode shows that metals extracted from the aqueous phase by using a mentioned ionic liquid, can be recovered in the metallic form which is one of the best ways of producing chemically pure metals for many different branches of industry. Moreover, to prove an easy regeneration of the ionic liquid five cycles of silver extraction and regeneration by using [eth-hex-en][Tf₂N] have been conducted. The initial concentration of silver in the aqueous solution was equal 0.05 M/L (about 5.4 g/L), the extraction process was designed to obtain 100% saturation of the ionic liquid, so some part of unextracted silver were left in the solution. However, the most important aspect of the study was to find if the overall capacity of the ionic liquid stays at the same level after the electro regeneration processes. The cyclic voltammograms in FIG. 10 show the feasibility of electroplating copper, lead, and silver from [eth-hex-en][Tf₂N].

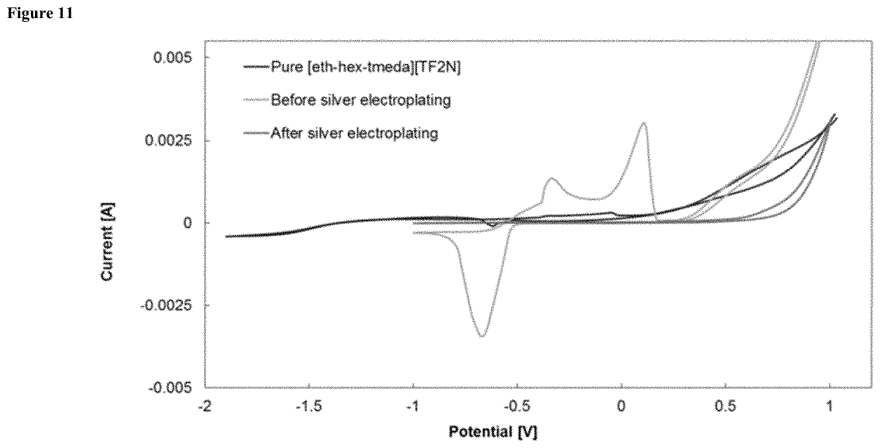

For the removal and electroplating of more reducing metals the electrochemical window of [eth-hex-tmeda][Tf₂N] was studied. FIG. 11 shows the CV of the pure IL. For the electroplating analysis, 10 mL of the IL was 20% saturated with silver, the CV of metal-IL complex is shown in FIG. 11, then the metal was electroplated onto a carbon electrode at constant potential of −0.75 V. The CV of the IL after the electroplating shown no silver reduction peaks, FIG. 11.

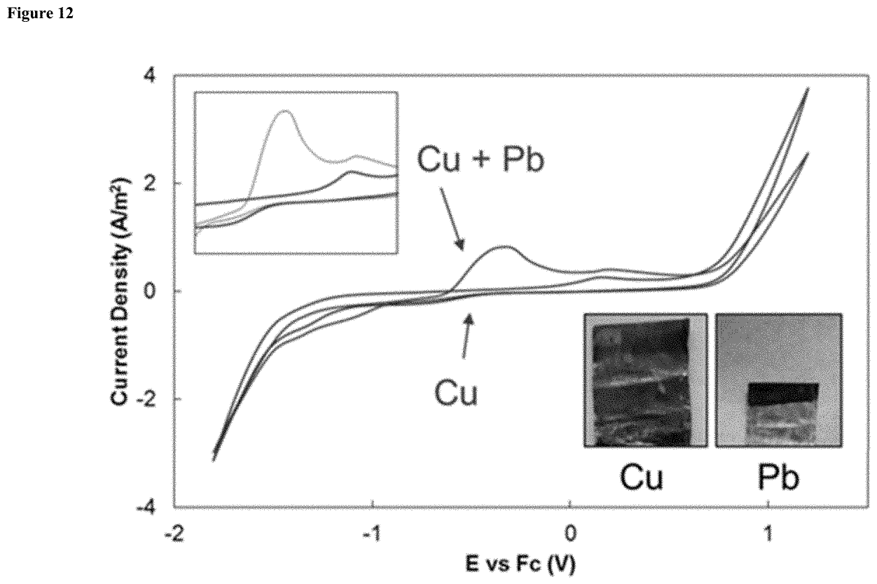

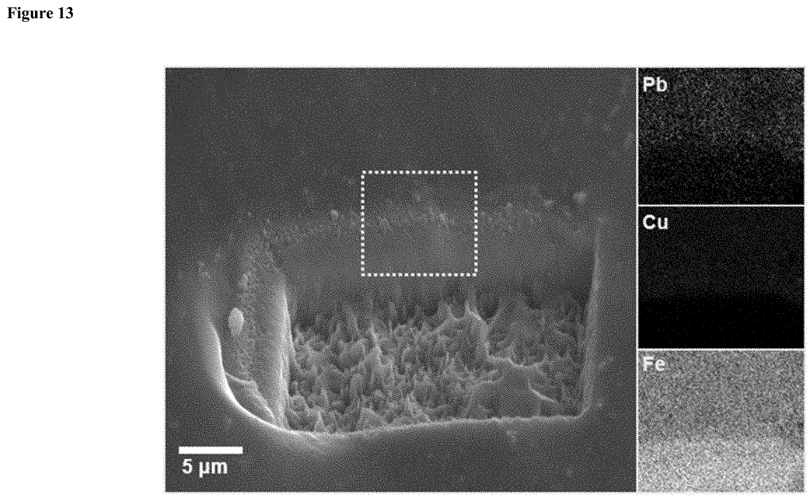

An important thrust of this work is that very selective separation of the metals can be used in two different steps, first at the stage of extraction, where some metals can be easily chelated and some others cannot. Another stage of selectivity in the separation process is the electroplating of metals from ILs onto the electrode. Each of the complexed metals can be recovered at a different electroplating potential, so it is possible to extract two different metals from the aqueous phase to the ionic liquid and then, plate each metal from the organic phase at a different potential. Analysis of the mentioned separation method was conducted by using an equimolar water solution of copper and lead nitrates. After the extraction process in which both copper and lead cations have been complexed by the [eth-hex-en][Tf₂N], a potential sweep was applied between the reduction potentials of copper and lead over a period of 12 hours. FIG. 12 shows the cyclic voltammograms of these complexes with the independently plated electrodes. FIG. 13 shows the elemental analysis of a sequentially coated electrode.

### Continuous Process of Removal with Sequential Removal of Metals

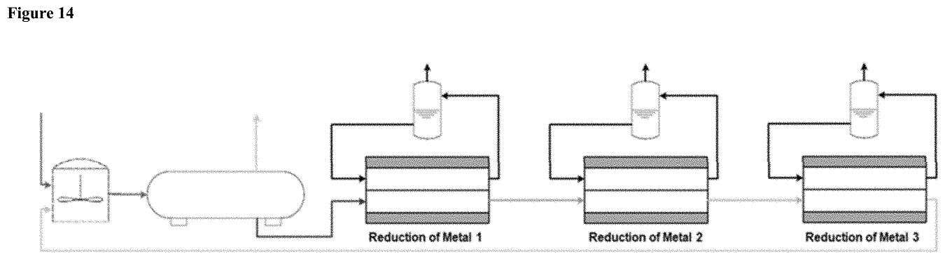

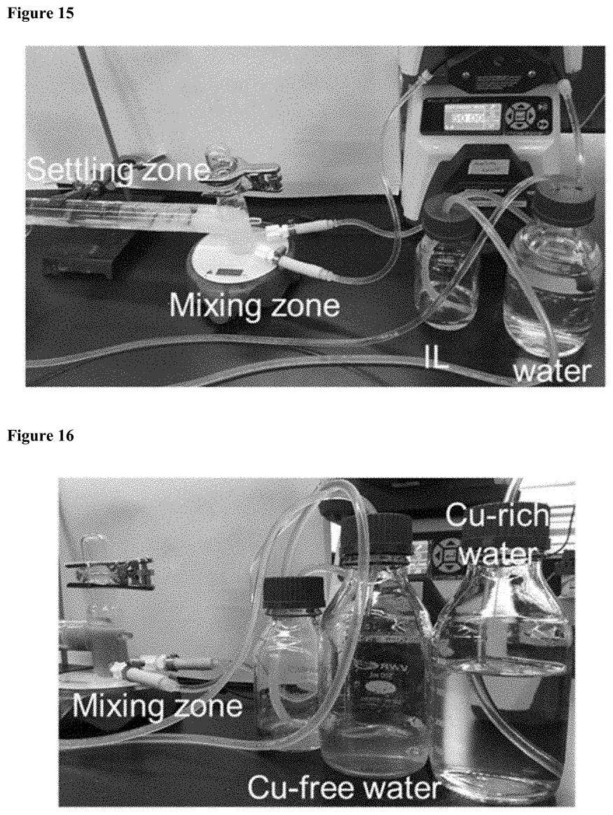

Thanks to the possibility of selective electroplating of the metals that are complexed in the ionic liquid it is also possible to use the ILs in this work for a sequence continuous removal and plating of metals at different stages of the process. That would allow for an excellent separation of the metals which is very often an important problem in many different industrial processes. Good examples of the process where this type of separation could be very useful are the mining processes, in which, there is more than one metal in the aqueous phase after the leaching process. Quite often metals present in the solution can have similar chemical properties and behavior, like e.g. copper and nickel mixture, so it is hard to separate them by using classical techniques. However, thanks to a huge difference in a reduction potential that separation can be easily achieved by using direct electroplating systems. Ionic liquids with wide electrochemical windows and low binding specificity can be mixed with a solution of many different metals and then after phase separation, metals can be plated directly from ionic liquids in electrochemical cells with increasing potential between the electrodes, adjusted precisely for selected metals. FIG. 14 shows a schematic of this proposed process. The mixer settler portion of this process was implemented to test the continuous extraction of the copper using [eth-hex-en][Tf₂N]. FIGS. 15 and 16 show the bench-scale continuous extraction system.

The system shown in FIG. 15 was prepared by using deionized water, to calculate and adjust flow rates of ionic liquid and water phase in the system. After the mixing zone, in which an extraction process is taking place, there is a long flow settler, which allows a phase separation after the extraction. The residence time of the liquids was adjusted to allow for complete phase separation.

FIG. 16 shows the system after 30 minutes of extraction. It can be seen that the aqueous phase containing copper from a bottle on the right has been purified by the system and transferred to a middle bottle in the form of clear pure water without any traces of metals. ICP analysis confirmed that copper removal in that system was 100%, similar to the batch extraction analyses.

In conclusion, the use of hydrophobic task specific ionic liquids with functional anions was demonstrated for use in waste water treatment and the recovery of metals. Known techniques of extraction into an ionic liquid phase was combined with the ability to electrochemically recycle the extraction medium. Extraction efficiency is greater than 99% with the resulting chelated ILs demonstrating large electrochemical windows suitable for fast metal deposition and recycling. It is also clear from the CVs that selective deposition may be applied to certain metal combination. Future approaches may consider polymeric analogues for flow systems, tuning of ionic liquids for control of physicochemical properties and the control of deposition.

### REFERENCE CITED

1. P. B. Tchounwou, C. G. Yedjou, A. K. Patlolla, D. J. Sutton, Heavy Metals Toxicity and the Environment, EXS, 2012, 101, 133-164.

2. H. Bradl, Editor. Heavy Metals in the Environment: Origin, Interaction and Remediation Volume 6. London: Academic Press, 2002.

3. S. Babel, T. A. Kurniawan, Various treatment technologies to remove arsenic and mercury from contaminated groundwater: an overview, Proceedings of the First International Symposium on Southeast Asian Water Environment, Bangkok, Thailand, 2003, 24-25, 433-440.

4. J. H. Duffus, Heavy metals-a meaningless term?, Pure Appl. Chem., 2002, 74, 793-807.

5. K. C. Kang, S. S. Kim, J. W. Choi, S. H. Kwon, Competitive adsorption characteristics of Co2+, Ni2+, and Cr2+ by IRN-77 cation exchange resin in synthesized wastewater, Chemosphere, 2004, 56, 131-147.

6. B. Alyüz, S. Veli, Kinetics and equilibrium studies for the removal of nickel and zinc from aqueous solutions by ion exchange resins, J. Hazard. Mater., 2009, 167, 482-488.

7. E. Sampera, M. Rodrigueza, M. A. De la Rubia, D. Prats, Removal of metal ions at low concentrations by micellar-enhanced ultrafiltration (MEUF) using sodium dodecyl sulfate (SDS) and linear alkylbenzene sulfonate (LAS), Sep. Purif., Technol., 2009, 65, 337-342.

8. Q. Chang, G. Wang, Study on the macromolecular coagulant PEX which traps heavy metals, Chem. Eng. Sci., 2007, 62, 4636-4642.

9. M. Plattes, A. Betrand, B. Schmitt, J. Sinner, F. Versraeten, J. Welfring, Removal of tungsten oxyanions from industrial wastewater by precipitation coagulation and flocculation processes, J. Hazard. Mater. 2007, 148, 613-615.

10. Barakat M. A., New trends in removing heavy metals from industrial wastewater, Arabian J. Chem., 2011, 4, 361-377.

11. R. Apiratikul, P. Pavasant, Batch and column studies of biosorption of heavy metals by Caulerpa lentillifera, Bioresour. Technol., 2008, 99, 2766-2777.

12. Y. Ku, I. L. Jung, Photocatalytic reduction of Cr(VI) in aqueous solutions by UV irradiation with the presence of titanium dioxide, Water Res., 2001, 35, 135-142.

13. M. M. Matlock, K. R. Henke, D. A. Atwood, Effectiveness of commercial reagents for heavy metal removal from water with new insights for future chelate designs, J. Hazard. Mater., 2002, 92, 129-142.

14. Y. Xu, F. Zhang, Experimental research on heavy metal wastewater treatment with dipropyl dithiophosphate, J. Hazard. Mater., 2006, 137, 1636-1642.

15. I. K. Wang, Y. T. Hung, N. K. Shammas, Advanced physicochemical treatment technologies. In: Handbook of Environmental Engineering, vol. 5, Humana, New Jersey, 2007.

16. M. L. Dietz, J. A. Dzielawa, Ion-exchange as a mode of cation transfer into room-temperature ionic liquids containing crown ethers: implications for the “greenness” of ionic liquids as diluents in liquid-liquid extraction, Chem. Commun., 2001, 2124-2125.

17. A. E. Visser, R. P. Swatloski, W. M. Reichhert, R. Mayton, S. Sheff, A. Wierzbicki, J. H. Davis Jr, R. D. Rogers, Task-specific ionic liquids for the extraction of metal ions from aqueous solutions, Chem. Commun., 2001, 135-136.

18. R. S. Kalb, Method for Producing Ionic Liquids, Ionic Solids or Mixtures thereof, 2005, WO2005/021484.

19. L. Fischer, T. Falta, G. Koellensperger, A. Stojanovic, D. Kogelnnig, M. Galanski, R. Krachler, B. K. Keppler, S. Hann, Ionic Liquids for extraction of metals and metal containing compounds from communal and industrial waste water, Water Res., 2011, 45, 4601-4614.

20. D. Depuydt, L. Liu, C. Glorieux, W. Dehaen, K. Binnemans, Homogeneous liquid-liquid extraction of metal ions with non-fluorinated bis(2-ethylhexyl)phosphate ionic liuqids having a lower critical solution temperature in combination with water, Chem. Commun., 2015, 51, 14183-14186.

21. M. A. Valdes Vergara, I. V. Lijanova, N. V. Likhanova, O. Olivares Xometl, D. Jaramillo Vigueras, A. J. Morales Ramirez, Recycling and recovery of ammonium-based ionic liquids after extraction of metal cations from aqueous solutions, Separation and Purif. Technol., 2015, 155, 110-117.

22. P. Zhang, Y. Gong, Y. Lv, Y. Guo, Y. Wang, C. Wang, H. Li, Ionic Liquids with metal chelate anions, Chem. Commun., 2012, 48, 2334-2336.

23. J. R. Harjani, T. Friscic, L. R. MacGillivray, R. D. Singer, Metal Chelate Formation Using a Task-Specific Ionic Liquid, Inorg. Chem, 2006, 45, 10025.

24. H. Heitzman, B. A. Young, D. J. Rausch, P. Rickert, D. C. Stepinski, M. L. Dietz, Fluorous ionic liquids as solvents for the liquid-liquid extraction of metal ions by macrocyclic polyethers, Talanta, 2006, 69, 527.

25. Y. Song, C. Cheng, H. Jing, Aza-Crown Ether Complex Cation Ionic Liquids: Preparation and Applications in Organic Reactions, Chem. Eur. J., 2014, 20, 12894-12900.

26. G. Wei, Z. Yang, C. Chen, Room Temperature ionic liquid as a novel medium for liquid/liquid extraction of metal ions, Analytica Chimica. Acta, 2003, 488, 183.

27. M. P. Jensen, J. Neuefeind, J. V. Beitz, S. Skanthakumar, L. Soderholm, Mechanism of Metal Ion Transfer into Room-Temperature Ionic Liquids: The Role of Anion Exchange, J. Am. Chem. Soc., 2003, 125, 15466.

28. D. Kogelnig, A. Stojanovic, M. Galanski, M. Groessl, F. Jirsa, R. Krachler, B. K. Keppler, Greener synthesis of new ammonium ionic liquids and their potential as extracting agents, Tetrahedron Lett., 2008, 49, 2782.

29. V. M. Egorov, D. I. Djigailo, D. S. Momotenko, D. V. Chernyshov, I. I. Torocheshnikova, S. V. Smirnova, I. V. Pletnev, Task-specific ionic liquid trioctylamonium salicylate as extraction solvent for transition metal ions, Talanta, 2010, 80, 1177.

30. J. L. Manzoori, M. Amjadi, J. Abulhassani, Ultra-trace determination of lead in water and food samples by using ioni cliuqid-based single drop microextraction-electrothermal atomic absorption spectrometry, Anal. Chim. Acta, 2009, 644, 48.

31. D. Si, K. Chen, J. Yao, H. Li, Structures and Electronic Properties of Lithium Chelate-Based Ionic Liquids, J. Phys. Chem. B., 2016, 120, 3904.

32. S. Platzer, M. Kar, R. Leyma, S. Chib, A. Roller, F. Jirsa, R. Krachler, D. R. MacFarlane, W. Kandioller, B. K. Keppler, Task-specific thioglycolate ionic liquids for heavy metal extraction: Synthesis, extraction efficacies and recycling properties, J. Haz. Mater., 2017, 324, 241-249.

33. Y. Song, C. Cheng, and H. Jing, “Aza-Crown Ether Complex Cation Ionic Liquids: Preparation and Applications in Organic Reactions,” Chem.—A Eur. J., vol. 20, no. 40, pp. 12894-12900, Sep. 2014.

34. Dwi Siswanta, Kazuhisa Nagatsuka, Hiroyuki Yamada, Kazuhiro Kumakura, Hideaki Hisamoto, Yushi Shichi, Kazuaki Toshima, and Koji Suzuki, “Structural Ion Selectivity of Thia Crown Ether Compounds with a Bulky Block Subunit and Their Application as an Ion-Sensing Component for an Ion-Selective Electrode,” 1996.

### Incorporation by Reference

All US and PCT patent application publications and US patents cited herein are hereby incorporated by reference in their entirety as if each individual publication or patent was specifically and individually indicated to be incorporated by reference. In case of conflict, the present application, including any definitions herein, will control.

### Equivalents

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of removing metal cations from an ionic liquid mixture, comprising:
providing an ionic liquid mixture comprising an ionic liquid (IL), wherein the IL comprises a metal-chelating group, and a plurality of metal cations; and
applying an electrical potential to the ionic liquid mixture, thereby removing from the ionic liquid mixture the plurality of metal cations.

2.-4. (canceled)

5. The method of claim 1, wherein applying the electrical potential causes the plurality of metal cations to be electrochemically reduced.

6. The method of claim 1, wherein applying the electrical potential causes the plurality of metal cations to be electrochemically reduced to metal atoms.

7. The method of claim 1 claim 1, wherein the metal cation has a charge of +1, +2, or +3.

8. The method of claim 7, wherein the metal cation is a cation of Ag, Ca, Cd, Co, Cr, Cu, Er, Fe, Hg, Mg, Mn, Nb, Ni, Pb, Pd, Sc, Sn, Sr, V, Zn, Ce, Dy, Er, Eu, Fe, Gd, Ho, La, Lu, Nb, Nd, Pm, Pr, Sm, Tb, Tm, Yb, or a mixture thereof.

9.-12. (canceled)

13. The method of claim 1, wherein the metal-chelating group is selected from the group consisting of an ethylaminediacetic acid moiety, a crown ether, a dithizone, a hydroxyquinoline, 2-thenoyltrifluoroacetone, a thiosalicylate, a salicylate, a thiocarbamate, a dithiocarbamate, an alkanolamine, a thioglycolate, an aza-crown ether, and a thia-crown ether.

14. The method of claim 1, wherein the metal-chelating group is an ethylaminediacetic acid moiety.

15. The method of claim 1, wherein the IL comprises

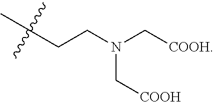 [SMILES: *CCN(CC(=O)O)CC(=O)O]

16. The method of claim 1, wherein the IL is

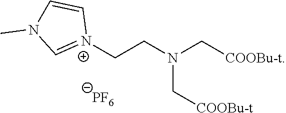

17. (canceled)

18. The method of claim 1, wherein the IL comprises a crown ether selected from the group consisting of 2-hydroxymethyl-12-crown-4; 12-crown-4; 15-crown-5; 2-aminomethyl-15-crown-5; 2-hydroxymethyl-15-crown-5; 4′-aminobenzo-15-crown-5; 24′-formylbenzo-15-crown-5; 4′-nitrobenzo-15-crown-5; 2,3-naphtho-15-crown-5; benzo-15-crown-5; dibenzo-15-crown-5; 18-crown-6; 2-aminomethyl-18-crown-6; benzo-18-crown-6; 2-hydroxymethyl-18-crown-6; 4′-aminobenzo-18-crown-6; dicyclohexano-18-crown-6; dibenzo-18-crown-6; 4′-aminodibenzo-18-crown-6; dibenzo-21-crown-7; dibenzo-24-crown-8; and dibenzo-30-crown-10.

19. (canceled)

20. The method of claim 1, wherein the IL comprises

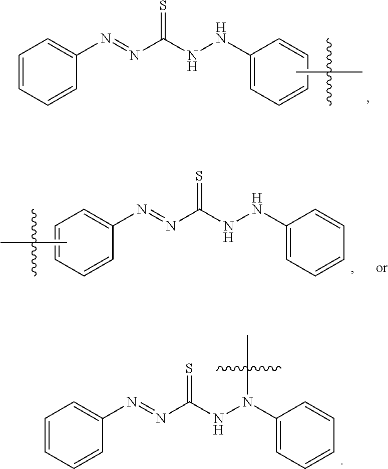

21. (canceled)

22. The method of claim 1, wherein the IL comprises

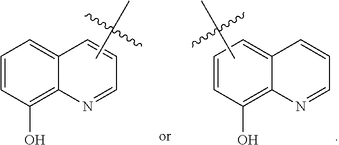

23. (canceled)

24. The method of claim 1, wherein the IL comprises

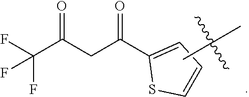

25. (canceled)

26. The method of claim 1, wherein the IL comprises

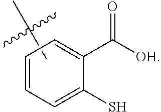

27. The method of claim 1, wherein the IL is trioctylammonium thiosalicylate or tricaprylmethylammonium thiosalicylate.

28. (canceled)

29. The method of claim 1, wherein the IL comprises

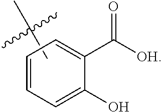

30. The method of claim 1, wherein the IL is trioctylammonium salicylate or tricaprylmethylammonium salicylate.

31. The method of claim 1, wherein the metal-chelating group is a thiocarbamate or a dithiocarbamate.

32. The method of claim 1, wherein the IL comprises

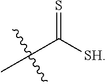 [SMILES: *C(=S)S]

33. The method of claim 1, wherein the IL comprises a dithiocarbamate selected from the group consisting of

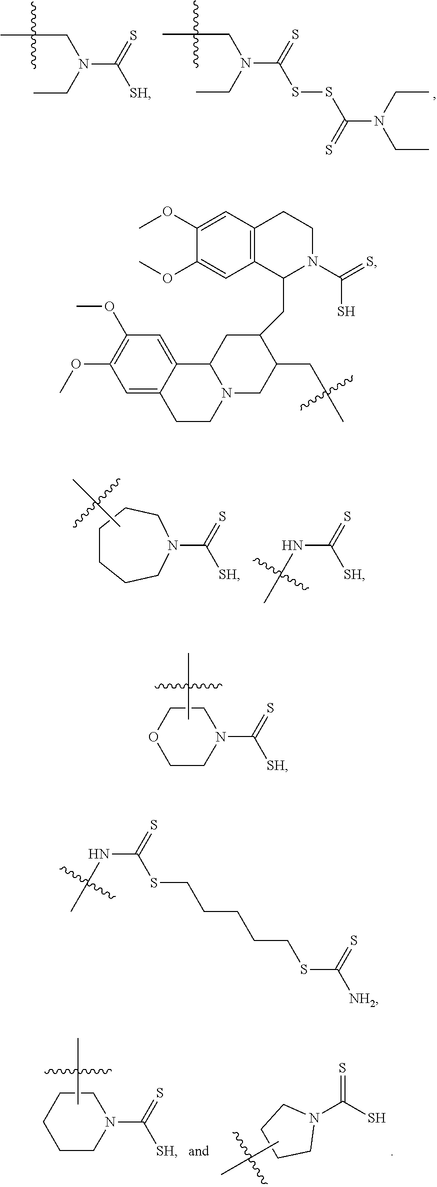

34. The method of claim 1, wherein the metal-chelating group is an alkanolamine.

35. The method of claim 1, wherein the IL comprises an alkanolamine selected from the group consisting of 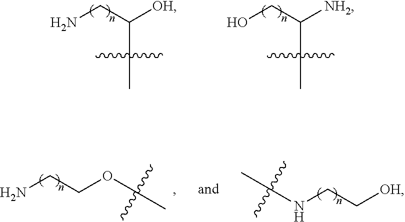
wherein n is an integer from 0 to 10.

36. The method of claim 1, wherein the IL is selected from the group consisting of monoethanolamine bis(trifluoroethanesulfonyl)amide, diethanolamine bis(trifluoroethanesulfonyl)amide, 2-((2-aminoethyl)amino)ethan-1-ol bis(trifluoroethanesulfonyl)amide, and 2,2′-(ethane-1,2-diylbis(oxy))bis(ethan-1-amine) bis(trifluoroethanesulfonyl)amide.

37. (canceled)

38. The method of claim 1, wherein the IL comprises a thioglycolate selected from the group consisting of

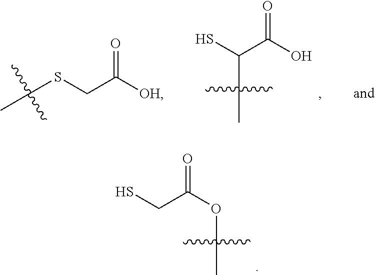 [SMILES: *SCC(=O)O ; *C(S)C(=O)O ; *OC(=O)CS]

39. The method of claim 1, wherein the IL is selected from the group consisting of methyltrioctylammonium butylsulfanyl acetate, methyltrioctylammonium pentylsulfanyl acetate, methyltrioctylammonium hexylsulfanyl acetate, methyltrioctylammonium benzylsulfanyl acetate, methyltrioctylphosphonium butylsulfanyl acetate, methyltrioctylphosphonium pentylsulfanyl acetate, methyltrioctylphosphonium hexylsulfanyl acetate, and methyltrioctylphosphonium benzylsulfanyl acetate.

40. (canceled)

41. The method of claim 1, wherein the IL comprises an aza-crown ether selected from the group consisting of 1-aza-12-crown-4; 1,7-diaza-12-crown-4; 4,10-diaza-12-crown-4; 1-aza-15-crown-5; 4,10-diaza-15-crown-5; N-phenylaza-15-crown-5; 1-aza-18-crown-6; 4,13-diaza-18-crown-6; 7,16-dibenzyl-1,4,10,13-tetraoxa-7,16-diazacycloocta-decane; 4,7,13,16,21-pentaoxa-1,10-diazabicyclo[8.8.5]tricosane; 4,7,13,16,21,24-hexaoxa-1,10-diazabicyclo[8.8.8]hexacosane; 5,6-benzo-4,7,13,16,21,24-hexaoxa-1,10-diazabicyclo[8.8.8]hexacos-5-ene; 5,6,14,15-dibenzo-1,4-dioxa-8,12-diazacyclopentadeca-5,14-diene; 1,4,10,13-tetraoxa-7,16-diazacyclooctadecane-1,4,7,10-tetraacetic acid; 1,4,7-triazacyclononane; 1,4,7-trimethyl-1,4,7-triazacyclononane; cyclen; hexacyclen; 1,4,7,10-tetraazacyclododecane; tri-tert-butyl 1,4,7,10-tetraazacyclododecane-1,4,7-triacetate; tri-tert-butyl 1,4,7,10-tetraazacyclododecane-1,4,7,10-tetraacetate; 1,4,8,12-tetraazacyclopentadecane; 1,4,8,11-tetraazacyclotetradecane; 1,4,8,11-tetraazacyclotetradecane-5,7-dione; and 1,4,8,11-tetramethyl-1,4,8,11-tetraazacyclotetradecane.

42. (canceled)

43. The method of claim 1, wherein the IL comprises a thia-crown ether selected from the group consisting of 1,4,7-trithiacyclononane; 3,6,9-trithia-1(2,5)-thiophenacyclodecaphane; 1,4,7,10-tetrathiacyclododecane; 1,4,8,11-tetrathiacyclotetradecane; 1,4,7,10,13-pentathiacyclopentadecane; 1,5,9,13-tetrathiacyclohexadecane; 1,5,9,13-tetrathiacyclohexadecane-3,11-diol; 1,4,7,10,13,16-hexathiacyclooctadecane; 2,3,5,6,8,9,11,12,14,15-decahydrobenzo[b][1,4,7,10,13,16]hexathiacyclooctadecine; hexabenzo[b,e,h,k,n,q][1,4,7,10,13,16]hexathiacyclooctadecine; and 1,4,10,13-tetrathia-7,16-diazacyclooctadecane.

44. The method of claim 1, wherein the ionic liquid comprises a cation and an anion; and the cation is represented by the following structural formula I: 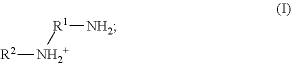
wherein, independently for each occurrence:
R¹ is —(C(R)₂)ₙ—;
n is 2, or 3;
R² is —(C(R′)₂)ₘ-R″;
m is 1, 2, 3, 4, 5, 6, 7, 8, 9, or 10; and
R is H, F, C₁-C₃ alkyl, or C₁-C₃ fluoroalkyl;
R′ is H, F, C₁-C₈ alkyl, or C₁-C₈ fluoroalkyl; and
R″ is H, F, C₁-C₃ alkyl, C₁-C₃ fluoroalkyl, C₁-C₃ alkyloxy, C₁-C₃ fluoroalkyloxy, C₆-C₁₀ aryl, C₂-C₈ alkenyl or C₂-C₈ fluoroalkenyl; wherein each instance of C₆-C₁₀ aryl is optionally substituted with one, two, three, four or five substituents independently selected from the group consisting of F, C₁-C₃ alkyl, C₁-C₃ fluoroalkyl, C₁-C₃ alkyloxy, and C₁-C₃ fluoroalkyloxy.

45. The method of claim 1, wherein the ionic liquid comprises a cation and an anion; and the cation is represented by the following structural formula II: 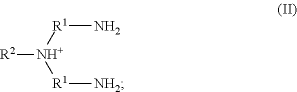
wherein, independently for each occurrence:
R¹ is —(C(R)₂)ₙ—;
n is 2, or 3;
R² is —(C(R′)₂)ₘ-R″;
m is 1, 2, 3, 4, 5, 6, 7, 8, 9, or 10; and
R is H, F, C₁-C₃ alkyl, or C₁-C₃ fluoroalkyl;
R′ is H, F, C₁-C₈ alkyl, or C₁-C₈ fluoroalkyl; and
R″ is H, F, C₁-C₃ alkyl, C₁-C₃ fluoroalkyl, C₁-C₃ alkyloxy, C₁-C₃ fluoroalkyloxy, C₆-C₁₀ aryl, C₂-C₈ alkenyl or C₂-C₈ fluoroalkenyl; wherein each instance of C₆-C₁₀ aryl is optionally substituted with one, two, three, four or five substituents independently selected from the group consisting of F, C₁-C₃ alkyl, C₁-C₃ fluoroalkyl, C₁-C₃ alkyloxy, and C₁-C₃ fluoroalkyloxy.

46. The method of claim 1, wherein an ionic liquid complex comprising an ionic liquid chelated to a metal cation is formed.

47. An ionic liquid complex, comprising an ionic liquid comprising a metal-chelating group chelated to a metal cation.

48.-79. (canceled)

80. An ionic liquid mixture comprising an ionic liquid and a metal-chelating group.

81.-112. (canceled)
